(19) United States
(12) Patent Application Publication
(10) Pub. No.: US 2019/0313903 A1
(43) Pub. Date: Oct. 17, 2019

(54) System and Method for Medical Condition Diagnosis, Treatment and Prognosis Determination

(71) Applicant: Big Picture Medical Proprietary Limited, Surry Hills (AU)

(72) Inventor: Tom Clarence McKinnon, Surry Hills (AU)

(21) Appl. No.: 16/464,670

(22) PCT Filed: Nov. 28, 2017
(86) PCT No.: PCT/AU2017/051313

§ 371 Date: May 28, 2019

(30) Foreign Application Priority Data
Nov. 28, 2016 (AU) 2016265973

(51) Int. Cl. A61B 3/14; A61B 3/12; A61B 3/10; G16H 70/60; G16H 50/20

(52) U.S. Cl. CPC A61B 3/14; A61B 3/12; G16H 50/20; G16H 70/60; A61B 3/102

(57) ABSTRACT

The apparatus and method disclosed relates to a system and method for identifying a medical condition in a patient. The system and method makes use of a remote terminal where tests and scans may be carried out and sent to a central server that receives patient medial data and detects anomalous characteristics in the tests and scans, and determines a diagnosis and probability of the diagnosis based on scans, tests presenting complaint and risk factors in the client medial history, lifestyle, or family medical history. Treatment and prognosis may also be determined in similar fashion. There is also provide an apparatus that simulates the effect of an ophthalmological condition on a virtual reality headset.

### FIELD OF THE INVENTION

The present disclosure relates to an eyecare system and method therefor.

The invention has been developed primarily for use in/with the eyes and will be described hereinafter with reference to this application. However, it will be appreciated that the invention is not limited to this particular field of use.

### BACKGROUND OF THE INVENTION

There are important benefits from obtaining regular eye examinations. Regular examinations enable practitioners to monitor and track the health of an individual's eyes and allows for early detection of diseases or disorders, or for early recognition of eye degeneration due to ageing, chronic disease (e.g., diabetes) or other relevant risk factors. Regular eye examinations also allow for a person's optical prescriptions to be kept up to date.

However, regular eye examinations may not be possible due to cost prohibitions, limited access to eye care professionals, or may be avoided due to the effort and spare time required in arranging for appointments at a medical practitioner or optometrist. What is needed is a system and method for ophthalmic condition recognition, care and prognosis determination which lessens problems associated with conventional systems.

### SUMMARY OF THE INVENTION

In a first aspect, there is provided a method of identifying a medical condition in a patient carried out on an electronic device, the method including the steps of receiving current medical data relating to the patient; detecting an anomalous characteristic in the current medical data; and determining a medical condition from the detected anomalous characteristic.

In one embodiment, the step of determining a medical condition includes the step of determining the probability of the detected anomalous characteristic being a medical condition.

In one embodiment, the step of determining a medical condition includes the step of detecting a plurality of anomalous characteristics in the current medical data.

In one embodiment, the step of receiving current medical data includes the step of receiving data from a digital ophthalmological data collection device configured for capturing data relating to a patient's eye.

In one embodiment, the digital ophthalmological data collection device is one or more selected from an optical coherency tomography (OCT) scanner; an adaptive optics scanning laser ophthalmoscopy (AOSLO) scanner; a scanning laser ophthalmoscopy (SLO) scanner; a mydriatic camera; a non-mydriatic camera; visual fields testing equipment; and intraocular pressure testing equipment.

In one embodiment, the step of detecting an anomalous characteristic includes the step of detecting a lesion in an image in the received current medical data.

In one embodiment, the step of detecting an anomalous characteristic includes the step of filtering the current medical data.

In one embodiment, the step of detecting an anomalous characteristic includes the step of filtering the current medical data by comparing the data to a characteristics of a healthy person.

In one embodiment, the instructions are configured for directing the processor to carry out the step of receiving patient data relating to the circumstances of the patient.

In one embodiment, the step of detecting an anomalous characteristic includes the step of filtering the current medical data by comparing the patient data to corresponding data of a healthy person.

In one embodiment, the step of detecting an anomalous characteristic includes the step of comparing at least part of the received current medical data to a control database of corresponding medical data of healthy people.

In one embodiment, the received patient data includes one or more selected from patient historical data; patient historical medical data; and patient family medical history data.

In one embodiment, the step of determining a medical condition includes the step of accessing a condition database listing any two or more selected from medical conditions, and anomalous characteristics associated with the medical condition; and risk factors associated with that medical condition, the presence of which increases the likelihood of the anomalous characteristic being indicative of that medical condition.

In one embodiment, the step of determining a medical condition includes the step of providing a condition database of ophthalmological conditions listing ophthalmological conditions, anomalous characteristics associated with the medical condition, and risk factors associated with that medical condition, the presence of which increases the likelihood of the anomalous characteristic being indicative of that medical condition.

In one embodiment, the step of determining a medical condition includes the step of comparing at least one or more detected anomalous characteristic of the patient to anomalous characteristics listed in the condition database to detect a match; and retrieving at least one or more medical condition associated with a matching anomalous characteristic.

In one embodiment, the condition database includes risk factors associated with at least one of the medical conditions, and the step of determining a medical condition includes the step of comparing at least one or more detected anomalous characteristics to the listed anomalous characteristics, comparing at least part of the information in the patient data to the risk factors associated with the listed anomalous characteristics to detect a matching risk factor; and retrieving at least one or more medical condition associated with a matching anomalous characteristic.

In one embodiment, the condition database includes risk factors associated with at least one of the ophthalmological conditions, and the step of determining a medical condition includes the step of comparing the detected anomalous characteristic and the patient data to the listed anomalous characteristics, and comparing the risk factors associated with the listed anomalous characteristics in the condition database to determine the probability of the detected anomalous characteristic being indicative of a listed medical condition associated with the listed anomalous characteristic.

In one embodiment, the step of determining a medical condition includes the step of directing the assimilation of a condition database of medical conditions, with associated anomalous characteristics and associated risk factors.

In one embodiment, the method includes the step of transmitting a diagnosis signal indicative of the results of the determination of the ophthalmological condition.

In one embodiment, the diagnosis signal includes one or more selected from the patient data; one or more detected anomalous characteristics; one or more the determined medical conditions associated with the one or more anomalous characteristics; the determined probability of the detected anomalous characteristics being indicative of the medical condition; and the patient data matched with associated risk factors influencing the determined probability.

In one embodiment, the instructions are configured for directing the processor to carry out the step of causing the display of one or more determined medical conditions.

In one embodiment, the instructions are configured for directing the processor to carry out the step of causing the display of one or more determined medical conditions together with the associated probability of the medical condition.

In one embodiment, the instructions are configured for directing the processor to carry out the step of causing the display of one or more determined medical conditions together with the matched risk factors used to determine the probability of the determined medical condition.

In one embodiment, the medical condition is an ophthalmological condition.

In one embodiment, the instructions are configured for directing the processor to carry out the step of receiving patient details uniquely identifying the patient; and storing the patient details in association with the patient's current medical data.

In one embodiment, the instructions are configured for carrying out the step of: receiving input from a medical practitioner confirming a that the determined medical condition is a correctly determined medical condition.

In one embodiment, the method includes the step of: retrieving management plan information for the correctly determined medical condition.

In one embodiment, the condition database includes management plan information associated with at least one or more medical conditions, and the instructions are configured for carrying out the step of retrieving management plan information from the condition database associated with one or more correctly determined medical conditions:

In one embodiment, the management plan information includes treatment scheduling information and the instructions are configured for carrying out the step of: scheduling treatment for the patient based on any one or more selected from the treatment scheduling information, the patient's schedule and the schedule of a medical practitioner.

In a further aspect, there is provided a system for identifying an abnormal medical condition in a patient carried out on an electronic device, the system including a processor; a network interface coupled to processor; digital storage media operatively associated to the processor, the digital storage media including: an anomalous characteristic detection module configured to receive current medical data relating to the patient, and detect an anomalous characteristic in the current medical data indicative of an anomaly that could be indicative of a medical condition; a medical condition determination module configured to compare the detected anomalous characteristic to a database of anomalous characteristics to retrieve an associated medical condition as a determined medical condition.

In one embodiment, the anomalous characteristic detection module is configured for receiving data from a digital ophthalmological data collection device configured for capturing current medical data relating to a patient's eye.

In one embodiment, the anomalous characteristic detection module is configured to filter the received current medical data to detect the anomalous characteristic.

In one embodiment, the anomalous characteristic detection module is configured to filter the patient's received current medical data against medical data of healthy patients.

In one embodiment, the anomalous characteristic detection module is configured to receive patient data relating to the circumstances of the patient.

In one embodiment, the received patient data includes one or more selected from patient historical data; patient historical medical data; and patient family historical medical data.

In one embodiment, the anomalous characteristic detection module is configured to interrogate a control database that stores personal data of healthy people, and associated baseline medical data of healthy people.

In one embodiment, the system includes a control database that stores personal data of healthy people, and associated baseline medical data of healthy people.

In one embodiment, the anomalous characteristic detection module is configured to interrogate the control database to compare at least one or more of the received patient details to the personal data of healthy people in order to compare like for like medical details, and then retrieving associated medical data of healthy people as a baseline filter to detect an anomalous characteristic in the patient data.

In one embodiment, the medical condition determination module is configured to access a condition database including a plurality of medical conditions, and anomalous characteristics associated with the medical condition.

In one embodiment, the condition database further includes risk factors associated with that medical condition, the presence of which increases the likelihood of the anomalous characteristic being indicative of that medical condition.

In one embodiment, the system includes a condition database.

In one embodiment, the medical condition determination module is configured to compare at least one or more detected anomalous characteristic of the patient to anomalous characteristics listed in the condition database to detect a match; and retrieve at least one or more medical condition associated with a matching anomalous characteristic.

In one embodiment, the medical condition determination module is configured to interrogate the condition database to compare at least one or more detected anomalous characteristics to the listed anomalous characteristics, compare at least part of the information in the patient data to the risk factors associated with the listed anomalous characteristics to detect a matching risk factor; and retrieve at least one or more medical condition associated with a matching anomalous characteristic.

In one embodiment, the system includes an assimilation direction module, the assimilation direction module being configured to directing the assimilation of a condition database of medical conditions, with associated anomalous characteristics and associated risk factors.

In one embodiment, the assimilation direction module is configured to direct the assimilation of a condition database in a networked supercomputer.

In one embodiment, the system includes a reporting module, the reporting module being configured for transmitting a diagnosis signal indicative of the results of the determination of the medical condition.

In one embodiment, the diagnosis signal includes information including any one or more selected from the patient details; the detected anomalous characteristic, the determined medical condition, the determined probability of the detected anomalous characteristic being indicative of the medical condition, the patient details matching the risk factors affecting the determined probability; and risk factors associated with the medical condition.

In one embodiment, the diagnosis signal includes information identifying a plurality of possible determined medical conditions, the determined probability of the detected anomalous characteristic being indicative of each of the possible determined medical conditions, and the patient details matching the risk factors affecting the determined probability of each of the possible determined medical conditions.

In one embodiment, the reporting module is configured to cause the display of one or more selected from the following: any of the patent details; one or more detected anomalous characteristics, one or more of the retrieved medical conditions associated with each anomalous characteristic, the probability of the detected anomalous characteristic being indicative of the medical condition; patient details matched with the risk factors that influence the probability of the anomalous characteristic being indicative of a medical condition; and risk factors associated with the medical condition.

In one embodiment, the reporting module is configured to receive confirmation of one or more retrieved medical conditions as being correctly determined.

In one embodiment, the system includes a scheduling module configured to retrieve management plan information associated with one or more correctly determined medical conditions.

In one embodiment, the management plan information includes treatment information indicative of the treatment required for treatment of the correctly determined medical condition.

In one embodiment, the management plan information is stored on the condition database.

In one embodiment, the scheduling module is configured for scheduling treatment of the patient in accordance with the management plan information with one or more selected from the patient; and a medical treatment provider.

In one embodiment, the medical condition is an ophthalmological condition.

In a further aspect, there is provided a system for identifying an abnormal medical condition in a patient carried out on an electronic device, the system including a processor configured for processing software instructions and configured for directing the transmission of signals from a transmitter; a receiver configured for receiving digital signals from a remote terminal, the receive being operatively connected to the processor to direct received signals to the processor for processing; a transmitter operatively connected to the processor and configured for transmitting signals as directed by the processor; and digital storage media configured for storing data and instructions configured for directing the processor to carry out the steps of: receiving current medical data relating to the patient; detecting an anomalous characteristic in the current medical data; and determining a medical condition from the detected anomalous characteristic.

In one embodiment, the step of determining a medical condition includes the step of determining the probability of the detected anomalous characteristic being a medical condition.

In one embodiment, the step of determining a medical condition includes the step of detecting a plurality of anomalous characteristics in the current medical data.

In one embodiment, the step of receiving current medical data includes the step of receiving data from a digital ophthalmological data collection device configured for capturing data relating to a patient's eye.

In one embodiment, the digital ophthalmological data collection device is one or more selected from an optical coherency tomography (OCT) scanner; an adaptive optics scanning laser ophthalmoscopy (AOSLO) scanner; a scanning laser ophthalmoscopy (SLO) scanner; a mydriatic camera; a non-mydriatic camera; visual fields testing equipment; and intraocular pressure testing equipment.

In one embodiment, the step of detecting an anomalous characteristic includes the step of detecting a lesion in an image in the received current medical data.

In one embodiment, the step of detecting an anomalous characteristic includes the step of filtering the current medical data.

In one embodiment, the step of detecting an anomalous characteristic includes the step of filtering the current medical data by comparing the data to a characteristic of a healthy person.

In one embodiment, the instructions are configured for directing the processor to carry out the step of receiving patient data relating to the circumstances of the patient.

In one embodiment, the step of detecting an anomalous characteristic includes the step of filtering the current medical data by comparing the data to a characteristic of a healthy person.

In one embodiment, the step of detecting an anomalous characteristic includes the step of comparing at least part of the received current medical data to a control database of corresponding medical data of healthy people.

In one embodiment, the received patient data includes one or more selected from patient historical data; patient historical medical data; and patient family medical history data.

In one embodiment, the step of determining a medical condition includes the step of accessing a condition database listing any one or more selected from medical conditions, anomalous characteristics associated with the medical condition; and risk factors associated with that medical condition, the presence of which increases the likelihood of the anomalous characteristic being indicative of that medical condition.

In one embodiment, the step of determining a medical condition includes the step of comparing at least one or more detected anomalous characteristic of the patient to anomalous characteristics listed in the condition database to detect a match; and retrieving at least one or more medical condition associated with a matching anomalous characteristic.

In one embodiment, the condition database includes risk factors associated with at least one of the medical conditions, and the step of determining a medical condition includes the step of comparing at least one or more detected anomalous characteristics to the listed anomalous characteristics, comparing at least part of the information in the patient data to the risk factors associated with the listed anomalous characteristics to detect a matching risk factor; and retrieving at least one or more medical condition associated with a matching anomalous characteristic.

In one embodiment, the step of determining a medical condition includes the step of directing the assimilation of a condition database of medical conditions, with associated anomalous characteristics and associated risk factors.

In one embodiment, the instructions are configured for directing the processor to carry out the step of transmitting a diagnosis signal indicative of the results of the determination of the medical condition.

In one embodiment, the diagnosis signal includes information identifying the determined ophthalmological condition.

In one embodiment, the diagnosis signal includes information identifying the determined medical condition and the determined probability of the detected anomalous characteristic being indicative of the medical condition.

In one embodiment, the diagnosis signal includes information identifying a plurality of determined possible medical conditions, and the probability of the detected anomalous characteristic being indicative of each of the possible medical conditions.

In one embodiment, the instructions are configured for directing the processor to carry out the step of causing the display of one or more determined medical conditions.

In one embodiment, the instructions are configured for directing the processor to carry out the step of causing the display of one or more determined medical conditions together with the associated probability of the medical condition.

In one embodiment, the instructions are configured for directing the processor to carry out the step of causing the display of one or more determined medical conditions together with the risk factors used to determine the medical condition.

In one embodiment, the medical condition is an ophthalmological condition.

In one embodiment, the instructions are configured for directing the processor to carry out the step of receiving patient details uniquely identifying the patient.

In one embodiment, the instructions are configured for carrying out the step of: presenting the diagnosed medical condition to a medical treatment provider.

In one embodiment, the step of presenting the diagnosed ophthalmological condition to a medical treatment provider includes the step of: presenting facts from the received patient data and current medical data as support for the determined probability of the determined of the ophthalmological condition.

In one embodiment, the instructions are configured for carrying out the step of: presenting several diagnosed ophthalmological conditions together with the probability of the determined medical conditions being correct.

In one embodiment, the instructions are configured for carrying out the step of: receiving input from a medical practitioner confirming at least one or more of the determined medical conditions as being a correctly determined medical condition; retrieving management plan information for the correctly determined medical condition.

In one embodiment, the condition data base includes management plan information associated with at least one or more medical conditions, and the instructions are configured for carrying out the step of retrieving management plan information from the condition database associated with one or more of the correctly determined medical conditions:

In one embodiment, the management plan information includes treatment scheduling information and the instructions are configured for carrying out the step of: scheduling treatment of the patient based on any one or more selected from the treatment scheduling information, the patient's schedule and the schedule of a medical treatment provider.

In a further aspect, there is provided a system for identifying an ophthalmological condition, including: a digital ophthalmological data collection device configured for capturing current medical data relating to a patient's eye a database of ophthalmological conditions including a plurality of condition profiles, each condition profile including at least two identifying characteristics of the condition; and a processor configured to: run the current medical data through a filter to detect abnormal ophthalmological characteristics; assign a weighting to each abnormal ophthalmological characteristic detected; and compare the weighted abnormal ophthalmological characteristics to the identifying characteristics in each condition profile in said database to identify an abnormal condition present in the digital image.

In one embodiment, said processor and said database are components of a web-based platform.

In one embodiment, said camera includes a microprocessor, said microprocessor being configured to receive a patient identification and associate the digital image with the patient identification.

In one embodiment, said camera includes a microprocessor, said microprocessor being configured to transmit only portions of the image containing each abnormal ophthalmological characteristic detected.

In one embodiment, said filter is generated based on a comparison with an image of a normal human eye.

In one embodiment, said filter is generated based on a comparison with an earlier ophthalmological image the same patient.

In one embodiment, said radio transmitter is configured as a Wi-Fi client.

In one embodiment, said radio transmitter is configured for peer-to-peer communications with a personal controller.

In one embodiment, said camera is configured as a mobile, hand-held ophthalmological camera.

In one embodiment, said wireless radio is configured for NFC communication.

In one embodiment, said wireless radio transmitter is configured as a GPS transmitter, said processor being configured to determine the geographic location of at least one eye specialist in close proximity to said camera.

In one embodiment, said processor is configured to utilise the abnormal condition identified in the image to match a patient having the abnormal identified condition with an eye specialist having a profile indicating experience in treating the abnormal condition, said processor being configured to send an eye specialist referral to the patient based on the match.

In a further aspect, there is provided a method for identifying an abnormal ophthalmological condition in a digital eye scan, including: producing, with a data collection device, the digital eye scan; passing the eye scan through a digital filter to detect at least one abnormal ophthalmological characteristic; assigning a weight to each abnormal ophthalmological characteristic detected; dynamically comparing the weighted characteristics detected with a plurality of characteristics indicative of abnormal ophthalmological conditions; and generating an ophthalmological condition report based on the dynamic comparison of weighted characteristics with indicative characteristics.

In one embodiment, the generation of the report includes assigning a risk percentage of the eye scan showing a specific abnormal ophthalmological condition.

In one embodiment, the risk percentage is calculated based on at least three weighted abnormal ophthalmological characteristics detected in the eye scan.

In one embodiment, the abnormal condition is an identified ophthalmological disease.

In one embodiment, the abnormal condition is an identified non-ophthalmological disease.

In a further aspect, there is provided a simulation system for simulating an ophthalmological condition, the system including: a camera for receiving an input and converting it into a visual image; a processor configured for processing data and instructions; digital storage media configured with instructions for directing a processor operationally; a headset configured for displaying the processed image on a headset display to a user on which the headset is mounted; the instructions being configured for interrogating a condition database of one or more ophthalmological conditions, each ophthalmological condition being associated with one or more image processing filters, the image processing filters being adapted to convert a visual image to a processed image, wherein the processed image simulates the effect of the ophthalmological condition on a person's vision when viewing that visual image.

In one embodiment, the system includes the condition database.

In one embodiment, the system includes an input device configured for receiving a condition selection input selecting one or more ophthalmological conditions to be simulated.

In one embodiment, the input device is configured for receiving a severity selection input selecting the severity of the ophthalmological condition to be simulated.

In one embodiment, the condition database includes severity manipulation information, the severity manipulation information being indicative of additional and/or alternatives processing required simulation of an ophthalmological condition depending on the severity selection input.

In one embodiment, the system includes a receiver for receiving one or more selected from the condition selection input and the severity selection input from a remote device.

In one embodiment, the system includes a transmitter for transmitting one or more selected from the condition selection input and the severity selection input to a remote device for interrogation of the condition database.

In one embodiment, one or more selected from the condition selection input and the severity selection input is provided as one or more floating point values that are used to determine the parameters to use for the image processing filters, and/or which image processing filters to use.

In one embodiment, the system includes an audio output device.

In one embodiment, the audio output device is configured to announce one or more selected from the ophthalmological condition and the severity of the phonological condition being displayed on the headset display.

In one embodiment, two or more image processing filters can be combined to simulate the effect of an ophthalmological condition.

In one embodiment, the instructions are configured for directing the processor to process the processed image for display on the headset display.

In one embodiment, the instructions are configured for directing the processor to process the processed image for display on the headset display as a pair of processed images.

As used herein, “configured” includes creating, changing, or modifying a program on a computer or network of computers so that the computer or network of computers behave according to a set of instructions. The programming to accomplish the various embodiments described herein will be apparent to a person of ordinary skill in the art after reviewing the present specification, and for simplicity, is not detailed herein. The programming may be stored on a computer readable medium, such as, but not limited to, a non-transitory computer readable storage medium (for example, hard disk, RAM, ROM, CD-ROM, USB memory stick, or other physical device), and/or the Cloud.

It will be appreciated that reference herein to “preferred” or “preferably” is intended as exemplary only.

It is to be understood that both the foregoing general description and the following detailed description are exemplary and explanatory only and are not restrictive of the invention, as claimed. In the present specification and claims, the word “comprising” and its derivatives including “comprises” and “comprise” include each of the stated integers, but does not exclude the inclusion of one or more further integers.

The claims as filed and attached with this specification are hereby incorporated by reference into the text of the present description.

The accompanying drawings, which are incorporated in and constitute a part of this specification, illustrate several embodiments of the invention and together with the description, serve to explain the principles of the invention.

This invention may also be said broadly to consist in the parts, elements and features referred to or indicated in the specification of the application, individually or collectively, and any or all combinations of any two or more of said parts, elements or features, and where specific integers are mentioned herein which have known equivalents in the art to which this invention relates, such known equivalents are deemed to be incorporated herein as if individually set forth.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

Other aspects of the invention are also disclosed.

### BRIEF DESCRIPTION OF THE DRAWINGS

Notwithstanding any other forms which may fall within the scope of the present disclosure, preferred embodiments of the invention will now be described, by way of example only, with reference to the accompanying drawings in which:

FIG. 1 shows a schematic view of a remote input terminal on which the various embodiments described herein may be implemented in accordance with an embodiment of the present disclosure;

FIG. 2 shows a schematic diagram of a remote input terminal, a service provider system and a user's remote terminal;

FIG. 3 shows a partial flowchart of a method of identifying a medical condition in a patient in accordance with one embodiment;

FIG. 4 shows a partial flowchart of a method of identifying a medical condition in a patient in accordance with another embodiment;

FIG. 5 shows top perspective view of a headset in a network with a user's mobile phone;

FIG. 6 shows a partial flowchart of a method of identifying a medical condition in a patient in accordance with further embodiment; and

FIG. 7 shows a schematic view of a simulation system on which various embodiments described herein may be implemented.

### DESCRIPTION OF EMBODIMENTS

Reference will now be made in detail to the present preferred embodiments of the disclosure, examples of which are illustrated in the accompanying drawings.

It should be noted in the following description that like or the same reference numerals in different embodiments denote the same or similar features.

### Remote Input Terminal

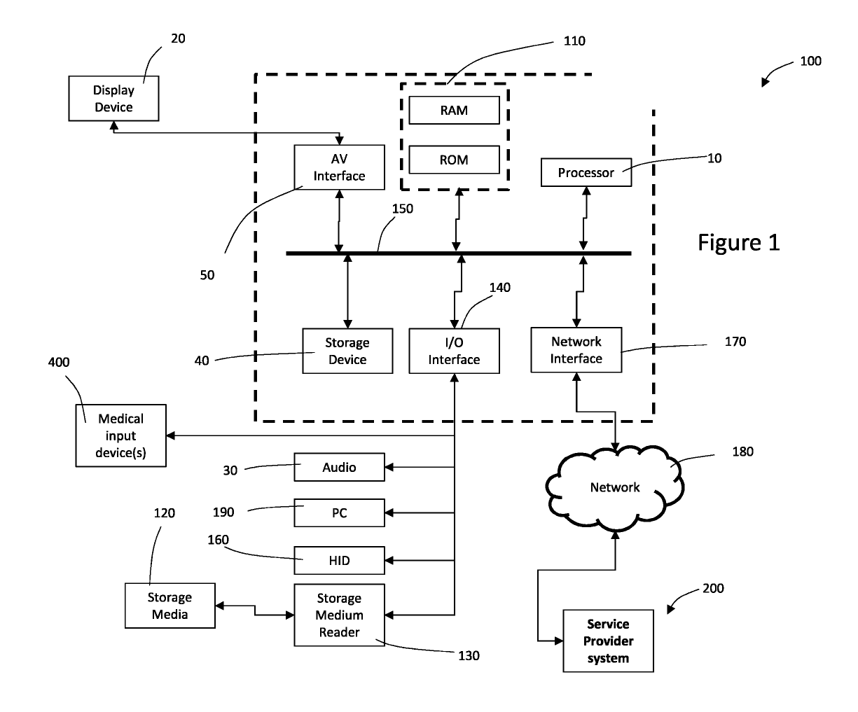

FIG. 1 shows a schematic view of a remote input terminal 100 on which the various embodiments described herein may be implemented. As will be apparent from the description below, the remote input terminal 100 is preferably mobile in nature and can be deployed in various embodiments for the purposes of receiving, recording, storing, processing and transmitting data relating to the current ophthalmological status of a patient. In one embodiment, the remote input terminal 100 can take the form of a web server and associated client computing device or the like depending on the application, however a dedicated machine is preferred.

In particular, the steps of the methodology described herein may be implemented as computer program code instructions executable by the remote input terminal 100. The computer program code instructions may be divided into one or more computer program code instruction libraries, such as dynamic link libraries (DLL), wherein each of the libraries performs one or more steps of the method. Additionally, a subset of the one or more of the libraries may perform graphical user interface tasks relating to the steps of the method.

The remote input terminal 100 includes semiconductor memory 110 including volatile memory such as random access memory (RAM) or read only memory (ROM). The memory 110 may include either RAM or ROM or a combination of RAM and ROM.

The remote input terminal 100 includes a computer program code storage medium reader 130 for reading the computer program code instructions from computer program code storage media 120. The storage media 120 may be optical media such as CD-ROM disks, magnetic media such as floppy disks and tape cassettes or flash media such as USB memory sticks or solid state disk (SSD).

The device further includes I/O interface 140 for communicating with one or more peripheral devices. The I/O interface 140 may offer both serial and parallel interface connectivity. For example, the I/O interface 140 may comprise a Small Computer System Interface (SCSI), Universal Serial Bus (USB) or similar I/O interface for interfacing with the storage medium reader 130. The I/O interface 140 may also communicate with one or more human input devices (HID) 160 such as keyboards, pointing devices, joysticks and the like for receiving input from a user.

The I/O interface 140 may also comprise a computer to computer interface, such as a Recommended Standard 232 (RS-232) interface, for interfacing the remote input terminal 100 with one or more personal computer (PC) devices 190. The I/O interface 140 may also comprise an audio interface for communicate audio signals to one or more audio devices 30, such as a speaker or a buzzer.

Further, the I/O interface can also comprise a visual interface for receiving signals from at least one or more medical input devices 400 as will be described in more detail below. In a preferred embodiment, the medical input device 400 is preferably one or more of an optical coherency tomography (OCT) scanner, an adaptive optics scanning laser ophthalmoscopy (AOSLO) scanner, a scanning laser ophthalmoscopy (SLO) scanner, a mydriatic camera, a non-mydriatic camera, visual fields testing equipment, and intraocular pressure testing equipment.

The remote input terminal 100 also includes a network interface 170 for communicating with one or more computer networks 180, thereby acting as both a transmitter and a receiver. The network 180 may be a wired network, such as a wired Ethernet™ network or a wireless network, such as a Bluetooth™ network or IEEE 802.11 network. The network 180 may be a local area network (LAN), such as a home or office computer network, or a wide area network (WAN), such as the Internet or private WAN.

The remote input terminal 100 includes an arithmetic logic unit or processor 10 for performing the computer program code instructions. The processor 10 may be a reduced instruction set computer (RISC) or complex instruction set computer (CISC) processor or the like. The remote input terminal 100 further includes a storage device 40, such as a magnetic disk hard drive or a solid-state disk drive.

Computer program code instructions may be loaded into the storage device 40 from the storage media 120 using the storage medium reader 130 or from the network 180 using network interface 170. During the bootstrap phase, an operating system and one or more software applications are loaded from the storage device 40 into the memory 110. During the fetch-decode-execute cycle, the processor 10 fetches computer program code instructions from memory 110, decodes the instructions into machine code, executes the instructions and stores one or more intermediate results in memory 100.

In this manner, the instructions stored in the memory 110, when retrieved and executed by the processor 10, can configure the remote input terminal 100 as a special-purpose machine that may perform the functions described herein.

The device 100 also includes a video interface 50 for conveying video signals to a display device 20, such as a liquid crystal display (LCD), cathode-ray tube (CRT) or similar display device.

The remote input terminal 100 also includes a communication bus subsystem 150 for interconnecting the various devices described above. The bus subsystem 150 may offer parallel connectivity such as Industry Standard Architecture (ISA), conventional Peripheral Component Interconnect (PCI) and the like or serial connectivity such as PCI Express (PCIe), Serial Advanced Technology Attachment (Serial ATA) and the like.

### Service Provider System

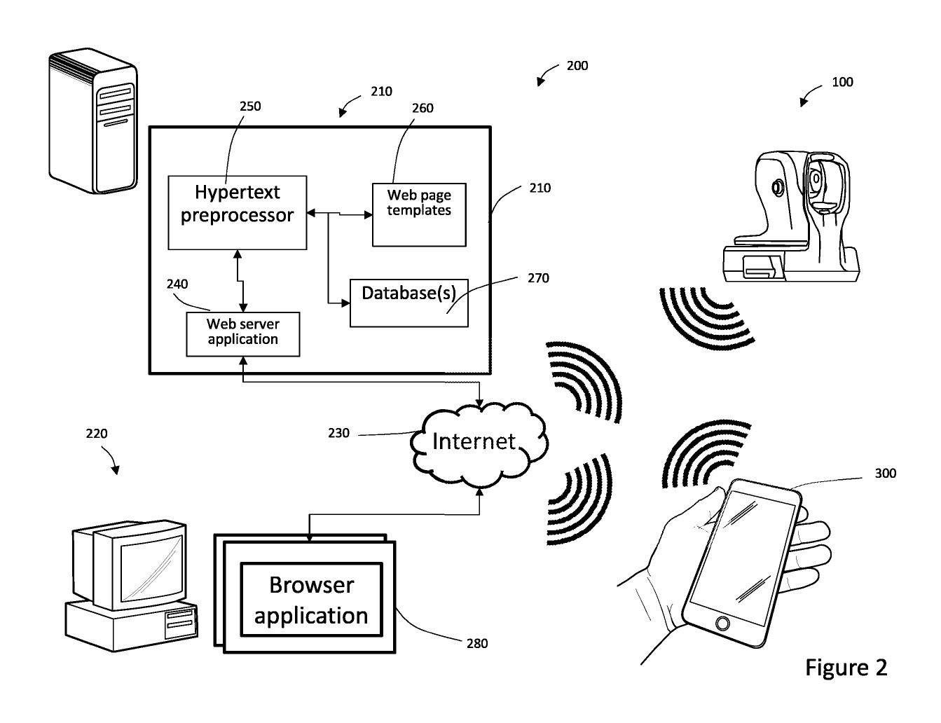

FIG. 2 shows a service provider system 200 on which an eyecare system for automated diagnosis of an ophthalmological condition in a patient can be implemented.

In a preferred embodiment, the computer methodology described herein is implemented by way of the service provider system 200 being networked with remote input terminals 100 communicating across the Internet 230 with the service provider system 200 utilizing web markup languages. However, should be noted that such deployment is one embodiment only and the computer methodology described herein may be implemented by other computing systems, networks and topologies.

The service provider system 200 includes a web server 210 for serving web pages to one or more client computing devices 220, mobile computing device 300 such as smart phones, and/or remote input terminals 100 over the Internet 230.

The web server 210 is provided with a web server application 240 for receiving requests, such as Hypertext Transfer Protocol (HTTP) and File Transfer Protocol (FTP) requests, and serving hypertext web pages or files in response. The web server application 240 may be, for example the Apache™ or the Microsoft™ IIS HTTP server, and is configured for receiving and transmitting information over a network including but not limited to the Internet.

The web server 210 is also provided with a hypertext preprocessor 250 for processing one or more web page templates 260 and data from one or more databases 270 to generate hypertext web pages. The hypertext preprocessor may, for example, be the Hypertext Preprocessor (PHP) or Microsoft Asp™ hypertext preprocessor. The web server 210 is also provided with web page templates 260, such as one or more PHP or ASP files.

Upon receiving a request from the web server application 240, the hypertext preprocessor 250 is operable to retrieve a web page template, from the web page templates 260, execute any dynamic content therein, including updating or loading information from the one or more databases 270, to compose a hypertext web page. The composed hypertext web page may comprise client side code, such as Javascript, for Document Object Model (DOM) manipulating, asynchronous HTTP requests and the like.

Client computing devices 220 are provided with a browser application 280, such as the Google Chrome™ Mozilla Firefox™ or Microsoft Internet Explorer™ browser applications. The browser application 280 requests hypertext web pages from the web server 210 and renders the hypertext web pages on a display device 20.

The service provider system 200 is also configured to transmit information to and receive information from mobile computing devices 300 such as smart phones. Such mobile computing devices may be owned or used by patients, and the service provider system may provide mobile enabled web pages, or an application (“app”) that is downloadable from app download facilities such as the Apple™ App Store or Google Play™

### Functionality

The functionality of the invention as it relates to the service provider system 200 and the remote input terminal 100 will now be described. It will be appreciated by those skilled in the art that any of the functionality ascribed to the service provider system 200 could also be carried out by the remote input terminal, and that any databases described below that are accessed and/or interrogated by the service provider system 200 could be accessed remotely by the remote input terminal 100.

### Remote Input Terminal Functionality

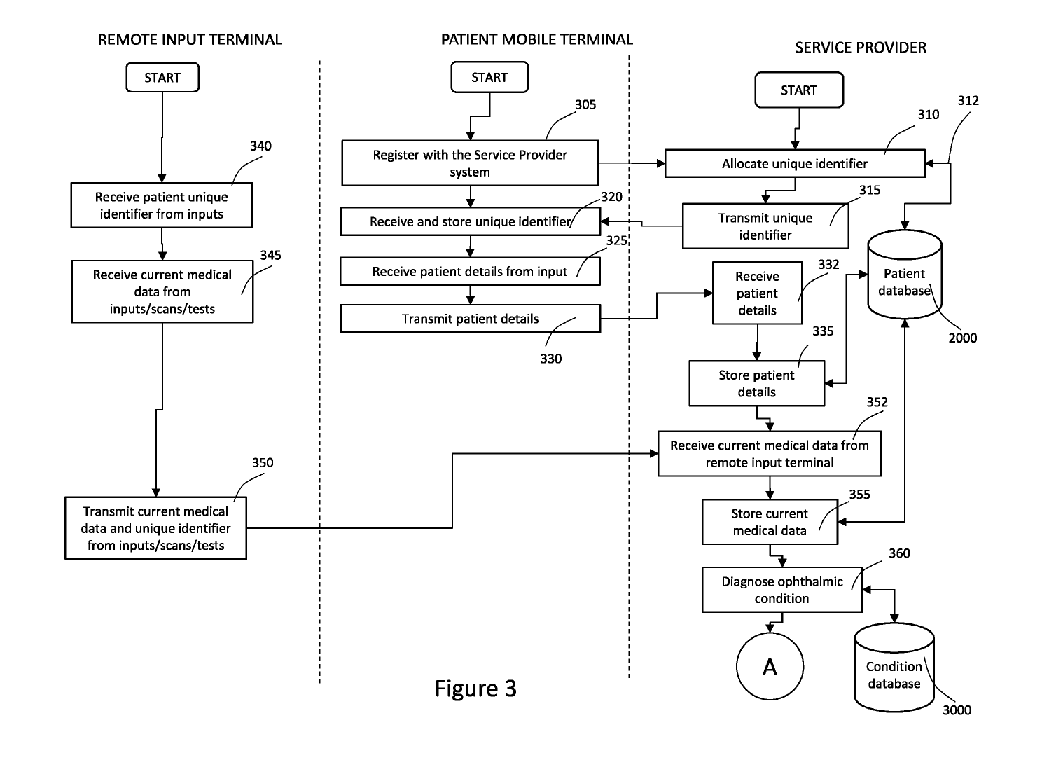

With reference to FIG. 3, it is envisaged that a patient can download an app on their smartphone, and may register 305 an account on a centralized database. The patient will be required to register 305 with the service provider system online, preferably providing proof of their identity, and will initially be allocated 310 a unique identifier, preferably in the form of a code or number. This unique identifier will be used in relation to any reports, diagnoses, inputs or transmissions, in order that such reports, diagnoses, inputs or transmissions are uniquely associated with that patient.

The patient will also be requested to input 325 relevant medical and/or non-medical details on their smartphone, which medical details are transmitted 330 to the service provider system, which are then received 332 for storage 335 the patient database in association with their unique identifier.

The non-medical details and medical details are transmitted 330 to the service provider system. The service provider system 200 includes a patient database 2000 of patients stored in association with their medical and non-medical details. The patient database 2000 is interrogated 312 to check whether the same patient has not previously registered. If similar patient names and details are found, the service provider system can generate an alert signal, so that this can be followed up. If no potential overlap of patients is found, then a unique identifier is generated for that patient, and stored in association with that patient's medical and non-medical details. The unique identifier is also transmitted 315 to the patient for their information, that will be transmitted 315 to the patient's mobile terminal, where it can be received 320 and stored in the app that is accessible by the patient to keep track of developments.

The patient's medical and/or non-medical details are described in more detail below, however they can include historical medical details for the patient and/or the patient's family. The patient will provide sufficient details for them to be uniquely identified in association with that patient.

It is then envisaged that a patient will go to a remote input terminal 100 that is conveniently located at optometrists, general medical practitioners, or even in more common location such as shopping malls or shopping centers.

Once the patient is registered with the service provider system 200, it is envisaged that the patient will attend the remote input terminal 100, at which current medical data indicative of the patient's current ophthalmological status can be obtained. It is envisaged that the remote input terminal 100 could include many different kinds of medical input devices.

The table below sets out tests, and output from the tests, that could be used as input for patient's current medical details:

| Tests/Equipment | Output |
|---|---|
| Slit-lamp (With digital camera) | Fundus Images |
| | Anterior Chamber Images (including angles) |
| | Corneal Surface Images |
| | External Eye Images (including lashes, lids etc) |
| Digital Photo | External Eye Images (including lashes, lids etc) |
| | Images of Skin Lesions |
| | Subconjunctival haemorrhage |
| | Red Eye |
| | Pterygium |
| | Discharge |
| Digital Video | External Eye Videos (including lashes, lids, pupil reactions, pupil movements, |
| | nystagmus etc) |
| | Cranial Nerve Assessments |
| | Assessment of throat lesions |
| Digital Otoscope | Tympanic membrane and external auditory canal images |
| Temperature Probe | Body Temperature (infective cause?) |
| Optical Coherence Tomography | 2D and 3D Anterior eye images (including cornea, anterior chamber +− lens) |
| | 2D and 3D Retinal images (including retina macula and optic nerve, thickness maps, |
| | comparison to normative data) |
| Digital Retinal Scan | 2D retinal images |
| Ultrawide Digital Retinal Scans | Ultrawide 2D Retinal Images |
| CT Scans Images | Eg Orbital fractures, intracranial pathology |
| MRI Images | Eg Orbital fractures, intracranial pathology |
| Xray Images | Eg Pulmonary pathology (eg with SLE) |
| Tonometers/Other IOP | Intraocular pressure (mmHg) |
| measurement devices | |
| Visual Fields Analysis (Digital) | Assessment of visual fields |
| Digital Video | Real time video conferencing with the patient |
| Keratometers | Measurement of the curvature of the anterior surface of the cornea (assessing |
| | astigmatism and keratoconus). |
| Autorefractometers | Assessment of Myopia, Hyperopia, Astigmatism, Anisometropia, Anisocoria, Gaze |
| | Deviation. |
| Point of care Hba1c | Measurement of hba1c (for diabetic patients) |
| Measurement | |
| Point of care Pregnancy test | Detection of hCG as an aid to early confirmation of pregnancy. |
| Point of care cholesterol | Measurement of blood total cholesterol, triglycerides, HDL, LDL and glucose (eg |
| measurement | Dyslipidaemia is a risk factor for AMD) |
| Point of care HIV test | Rapid HIV-1/2 Antibody Test detects antibodies to HIV-1 and HIV-2 |
| Blood pressure measurement | Measurement of systolic and diastolic blood pressure. (eg persons with hypertension |
| | are 1.5 times more likely to develop wet macular degeneration) |
| Body Mass Index (BMI) Derived | Obesity being obese may increase the chance that early or intermediate macular |
| from patient weight & height | degeneration progress to a more severe form of the disease. |
| Scanning Laser | SLO is a method of examination of the eye that uses the technique of laser scanning |
| Ophthalmoscope (SLO) | microscopy for diagnostic imaging of retina or cornea of the human eye. |
| Adaptive Optics Scanning Laser | AOSLO is a technique used to measure living retinal cells. It utilizes adaptive optics to |
| Ophthalmoscopy (AOSLO) | remove optical aberrations from images obtained from scanning laser ophthalmoscopy |
| | of the retina. |
| Blood tests | Eg for CMV, HIV, Leukaemia, Dyslipidaemia |
| Doppler OCT | Doppler OCT (DOCT), aims to visualize and quantify blood flow. |
| B-Scan | B-scan, is used to produce a two-dimensional, cross-sectional view of the eye and the |
| | orbit. It is commonly used when media opacity is present (Cataract, corneal opacity, |
| | vitreous bleed) |
| Contrast sensitivity test | A contrast sensitivity test measures a patient's ability to distinguish between finer and |
| | finer increments of light versus dark |
| Corneal topography | Corneal topography (also known as photokeratoscopy or videokeratography) is a non- |
| | invasive medical imaging technique for mapping the surface curvature of the cornea, the |
| | outer structure of the eye. |
| Vertometry | Vertometry (also known as a lensmeter or lensometer, focimeter or vertometer) is an |
| | ophthalmic instrument. It is mainly used by optometrists and opticians to verify the |
| | correct prescription in a pair of eyeglasses, to properly orient and mark uncut lenses, |
| | and to confirm the correct mounting of lenses in spectacle frames. |
| Tear osmolarity test | Testing for tear film hyperosmolarity (an indication of “Dry Eye”) |
| Optical biometry | Optical biometry is the current standard for intraocular lens (IOL) power calculations in |
| | clinical practice. Optical biometry is a highly accurate non-invasive automated method |
| | for measuring the anatomical characteristics of the eye. |

Examples of medical details would be current conditions and/or symptoms, medicines that they are taking currently, and the names and contact details of their doctor and/or specialist. Examples of non-medical details can include their name, age, address, contact details, insurance details, closest family, or the like.

The relevant medical details that the patient would be required to input can include historical medical details.

The table below sets out examples of current and historical medical details, as well as examples of typical questions that may be asked by the service provider that will assist in locating the presence of risk factors:

| Structured Opthamalic History | Questions |
|---|---|
| Presenting Complaint | Red eye |
| (PC) | Loss of Vision |
| | Lump |
| | Dry/Gritty Eyes |
| | Blurred or double vision |
| | Pain |
| | Flashes and floaters |
| History of Presenting | Red eye: |
| Complaint (HPC) | Sticky? Blepharitis, bacterial conjunctivitis |
| | Watery? Allergic conjunctivitis |
| | Painful? Anterior uveitis, angle closure glaucoma, scleritis, corneal ulcer |
| | Painless? Subconjunctival haemorrhage |
| | Vision Loss? Anterior uveitis, angle closure glaucoma, scleritis, corneal |
| | ulcer |
| | Normal Vision? Subconjunctival haemorrhage |
| | Duration Hours? Acute glaucoma, subconjunctival haemorrhage |
| | Duration Days? Uveitis, scleritis, episcleritis, corneal ulcer, conjunctivitis |
| | Loss of Vision |
| | Sudden? Ischaemic optic neuropathy, retinal artery/vein occlusion, optic |
| | neuritis |
| | Gradual? Cataract, ARMD, diabetic retinopathy |
| | Painful? Optic neuritis, angle closure glaucoma |
| | Painless? ARMD, retinal detachment |
| | Transient? TIA |
| | Permanent? Ovular infarct |
| | One eye? Ischaemic optic neuropathy, optic neuritis, glaucoma |
| | Both Eyes? ARMD, Diabetic maculopathy, bilateral optic nerve |
| | compression |
| | Lump (To be completed) |
| | Dry/Gritty Eyes (To be completed) |
| | Blurred or double vision (To be completed) |
| | Pain (To be completed) |
| | Flashes and floaters (To be completed) |
| | EXAMPLES OF OTHER QUESTIONS RELATED TO HPC |
| | Can have the patients subjectively grade any of the symptoms based on a |
| | sliding scale (eg 1-10) which is useful in triaging but also response to treatment |
| | Eyelid(s) affected? (e.g. blepharitis, entropion, ectropion, trichiasis) |
| | Periorbital swelling? (may suggest orbital cellulitis if other features are present |
| | like pain, reduced eye movements and systemic upset/pyrexia. Preseptal |
| | cellulitis presents with periorbital swelling but eye movements are not impaired.) |
| | Worse with eye movements? (scleritis) |
| | Dry or gritty? (e.g. keratoconjunctivitis, blepharitis) |
| | Sticky eye? (e.g. bacterial conjunctivitis, blepharitis) |
| | Is there an exudate? (presence, amount, colour) |
| | Is the eye watering? (keratitis, iritis, allergic conjunctivitis) |
| | Is there any photophobia? (iritis, keratitis, glaucoma) |
| | Painful? (Anterior uveitis, angle closure glaucoma, scleritis, corneal ulcer) |
| | Is there a glare in sunlight or difficulty driving at night due to the glare from |
| | headlights? (cataracts) |
| | Is the vision impaired? (multiple causes including glaucoma, cataract, uveitis, |
| | etc) |
| | Are there any floaters/flashes/haloes? (symptoms of retinal disease) |
| | Is there a headache with it? (pituitary tumours causing bitemporal hemianopia) |
| | Is there any urethral discharge? (Reiter's syndrome) |
| | History of foreign body insertion or trauma? |
| | Any eye itching or seasonal variation? |
| | Anyone in the family have similar eye problems? (e.g. transmission of viral |
| | conjunctivitis can occur from sharing towels) |
| Past Ophthalmic | Poor vision since birth or during childhood |
| History | History of lazy eye/amblyopia |
| | Recurrent ocular problems, particularly inflammatory (iritis) and herpes simplex |
| | keratitis |
| | Problems associated with contact lens wear (e.g. bacterial keratitis). Check for |
| | overwear (using daily wear contact lenses for more than 1 day) and if the |
| | correct contact lens solution is used. |
| | Recent cataract surgery (to look for complications of surgery such as |
| | endophthalmitis, wound infection, intraocular lens displacement causing a |
| | sudden drop in visual acuity) |
| | Past or recent refractive/corrective eye surgery |
| | Previous history of trauma to the eye (associated with cataract, glaucoma, |
| | retinal detachment) |
| | Ask if the patient has had a recent sight test to exclude an uncorrected |
| | refractive error. Myopia has been associated with retinal detachment and early |
| | onset vitreous degeneration, while hypermetropia has been associated with |
| | increased risk of acute angle closure glaucoma and pseudo papilloedema. |
| Past Medical History | Hypertension - associated with retinopathy, retinal vein occlusion |
| | Diabetes - associated with retinopathy, maculopathy, retinal and vitreous |
| | haemorrhage |
| | Systemic inflammatory disease eg sarcoidosis - may be associated with ocular |
| | inflammation (uveitis) |
| | History of ankylosing spondylitis (uveitis), connective tissue disorder (scleritis), |
| | inflammatory bowel disease, psoriasis, thyroid eye disease (ophthalmoplegia, |
| | diplopia), myasthenia gravis (ptosis) |
| | Cerebrovascular disease (CVA) |
| | History of dermatological conditions such as seborrhoeic dermatitis, atopic |
| | eczema, acne rosacea (all are strongly associated with anterior/posterior |
| | blepharitis) |
| | History of hay fever (atopy) |
| | Previous herpes infection on the face (herpetic eye disease) |
| | Previous history of immunosuppression (TB, HIV) |
| | Race (eg Whites are much more likely to lose vision from age-related macular |
| | degeneration than are Blacks or African-Americans). |
| Medications | List medications |
| | Allergies (required to know if prescribing) |
| | Amiodarone (corneal deposits (vortex keratopathy) |
| | Antiepileptics (Ocular motility dysfunction |
| | Benztropine, atropine (pupillary dilation -thus risk of angle closure glaucoma) |
| | Corticosteroids (Cataract, glaucoma) |
| | Digitalis (abnormal colour) |
| | Ethambutol, quinine (Optic neuropathy) |
| | Hydroxychloroquine, Chloroquine (Retinal degeneration change(“Bull's eye” |
| | macula) |
| | Opiates (Pupillary constriction |
| | Phenothiazines (Retinal oedema, pigmentary retinopathy, ocular motility |
| | dysfunction |
| | Sulfonamides, NSAIDS (Steven-Johnson Syndrome) |
| | Tamoxifen (pigmentary retinopathy) |
| Family History | Family history of ocular problems such as glaucoma, and ocular diseases that |
| | are known to be inherited such as retinitis pigmentosa. |
| | Family history of strabismus, refractive errors or amblyopia can help the |
| | diagnosis when faced with a child presenting with a squint. |
| | Family history of albinism, a group of inherited abnormalities of melanin |
| | synthesis. There are two types: ocular albinism (X-linked and recessive forms) |
| | associated with lack of pigmentation confined to the eye; and oculocutaneous |
| | albinism (recessive) where the hair and skin are also affected. |
| | Other ophthalmological conditions that have less well-defined associations |
| | include presenile cataract, retinal/corneal dystrophies and retinal detachment. |
| | The juvenile macular dystrophies are also a group of rare inherited conditions |
| | affecting the retinal pigment epithelium and photoreceptors. |
| | Family history of diabetes, hypertension, etc. |
| | Chances of developing age-related macular degeneration are three to four |
| | times higher if the patient has a parent, child, or sibling with macular |
| | degeneration. |
| Social History | Occupation (carpenter, boilermaker more susceptible to ocular foreign bodies) |
| | Smoking |
| | Alcohol |
| | Sexual history can help in some diagnoses eg Reiter's syndrome (uveitis or |
| | conjunctivitis). |

A person skilled in the art will appreciate that a wide variety of medical and non-medical risk factors can influence the diagnosis, and the above table is not intended to be an exhaustive list.

It is envisaged that the service provider will provide a simple list of questions to be answered by the patient. By providing a list of questions that can be answered, for example by selecting checkboxes on an electronic form, such questions can be accurately input by non-specialist and/or non-medically trained staff members. In this way, a structured history can be input for later use.

In addition, patients may be asked to grade the severity of symptoms or effects.

On testing the patient using any of the above medical input devices, the results of the tests are then transmitted 350 as current medical data to the service provider system 200, where it will be processed as will be discussed in more detail below.

This information will be received 345 by the remote input terminal 100, and transmitted 350 to the service provider as current medical data indicative of the patient's current medical status.

The received 352 current medical tightened will be stored 355 on the patient database. The patient's ophthalmological condition will then be diagnosed 360 by cross-referencing the patient details (including the current medical data, historical medical data and family historical medical data of the patient) against a condition database.

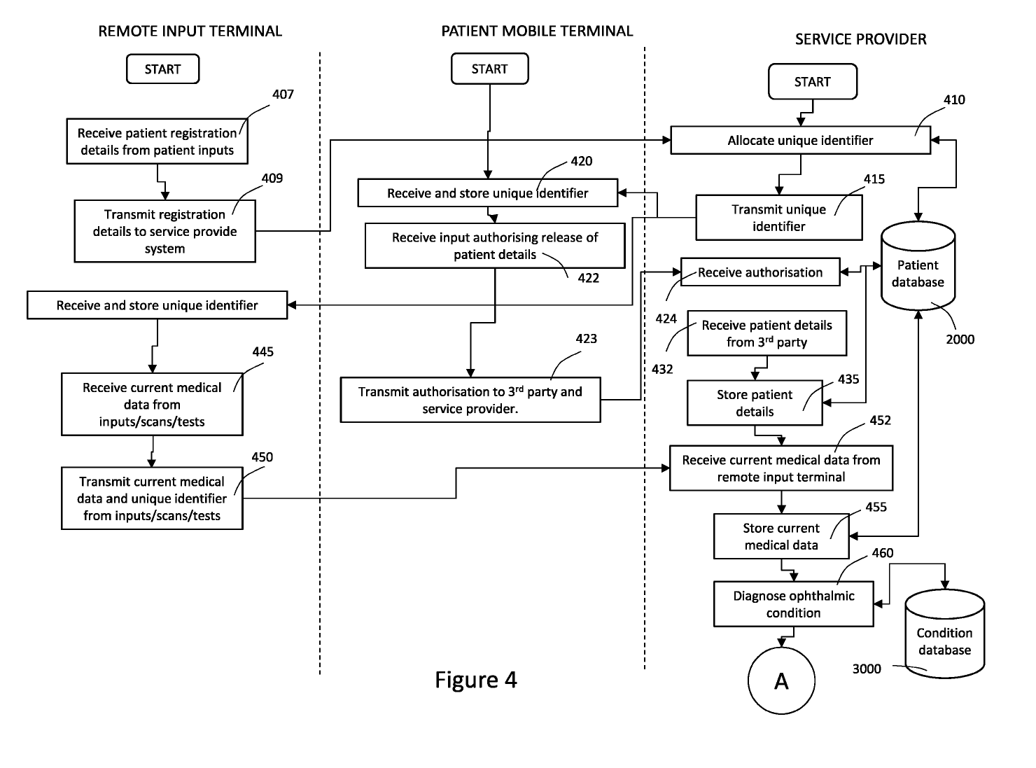

Alternately, as shown in FIG. 4, a patient can register 407 at and input relevant medical or non-medical details directly at the remote input terminal 100, preferably via a keyboard or touch enabled screen associated with the remote input terminal. Such details will also preferably include contact details for the patient's mobile terminal. These are then transmitted 409 to the service provider, where the details used to allocate 410 a unique identifier for that patient. The unique identifier is transmitted 415 to the patient's mobile terminal, where they will be preferably received and stored 420.

The patient will be asked to provide input authorising 422 the release of the details, for example from a third-party provider such as their optometrist or general practitioner. This authorisation will be transmitted 423 to the service provider, as well as to the third-party. The authorisation will be received 424 by the service provider and stored on the patient database.

On receiving this authorization, it is envisaged that the service provider system 200 can connect with the third-party provider to retrieve the patient's medical details. This may be retrieved in an automated, semi-automated or may be input manually. Thereafter, the service provider system will receive 432 patient details from the third-party, which will be stored 435 in the patient database.

It will be appreciated that the retrieval of patient details from third party providers can be carried out at any stage. For example, the patient details can be retrieved after an initial diagnosis is determined, or before any testing is carried out.

The unique identifier will be transmitted to the remote terminal 100, where it will receive 445 current medical data from a variety of inputs, scans and tests, and transmit 450 this current medical data to the service provider system 200 in association with the unique identifier.

In another embodiment (not shown), is envisaged that the remote input terminal 100 could interrogate the patient database 2000 for information stored in association with the patient's unique identifier, including medical details.

The remote input terminal can be configured for diagnosing an ophthalmological condition utilizing both the received historical medical details and the current medical data obtained from the medical input devices in much the same way as the service provider system can diagnose an ophthalmological condition as will be described in more detail below.

Is envisaged that the remote input terminal 100 can be further configured for, once a diagnosis has been determined, transmitting the results of the diagnosis to a medical treatment provider or service provider system 200 as will be discussed in more detail below. However, in one preferred embodiment, it is envisaged that the amount of data transmitted can be reduced in accordance with the diagnose ophthalmological condition. Specifically, the remote input terminal may be configured for determining a relevant portion of the input data that has been received from the medical input devices and/or tests, as well as determining a non-relevant portion of the input data. This determination is preferably carried out in accordance with the diagnosed ophthalmological condition, but could also be carried out from an initial screening of the input data based on only certain input such as visual images.

The non-relevant portion of the input data can be processed so that the amount of data to be transmitted is reduced, preferably while leaving sufficient contextual detail in the non-relevant data to allow a medical practitioner to understand the context of relevant portion of the input data. The relevant portions of the input data are preferably transmitted with as much detail as possible, thereby allowing further diagnosis and/or confirmation by the service provider system 200 and/or medical practitioner. The relevant portions, or all portions if desired, may be compressed and, or encrypted prior to transmission in order to facilitate transmitting speeds over data lines.

Preferably, the relevant portion of the input data together with the screened non-relevant portion of the input data is transmitted together.

It is envisaged that in one embodiment, only the relevant portion of the input data may be transmitted, however this is not preferred.

In this way, the amount of data to be transmitted from the remote input terminal 100 can be reduced, without losing any of the detail that may be required for a confirmatory diagnosis or further investigation.

### Service Provider Functionality

As mentioned previously, current medical data that has been input from the medical input devices or received from tests described above will be transmitted to, and received by the service provider system 200. The transmission of current medical data can include screened non-relevant portions of the current medical data as well as preferably unscreened relevant portions of the current medical data.

Once the current medical data is received by the service provider system 200, it is envisaged that provider system 200 will utilize the current medical data, together with the received historical medical details for the patient, and any other patient details, in order to identify the probability of an ophthalmological condition in that patient. The process of identification, which could be carried out by the service provider system 200, or by the remote input terminal 100 as described in more detail below.

It is envisaged that once the diagnosis has been carried out, the service provider system 200 will transmit the results of the identification of the medical condition to a medical practitioner.

### Identification of Medical Condition

As described above, it is envisaged that both the remote input terminal 100 or the service provider system 200 can carry out an automated condition identification process using the current medical data together with the received historical medical details for the patient and any other patient details. It will be appreciated by those skilled in the art that the process of identifying and determining the probability of an ophthalmological condition will be influenced by past events, patient medical history, and current medical data for that patient.

For example, a retinal scan may produce a visual image, from that visual image, a visual anomalous characteristic may be able to be detected. However, without further background information, no accurate diagnosis can be made. Even after a visual anomalous characteristic is detected, a diagnosis based on this visual anomalous characteristic can vary widely dependent, for example on whether or not the patient is known to be a diabetic, whether or not they have per levels of visual acuity, or whether the intraocular pressure in the eyeball is high.

The present disclosure takes these factors into account, by providing diagnostic algorithms that take into account the patient's prior medical history and other patient details when assessing the current medical data of the patient.

It is further envisaged that the diagnostic algorithms will further be configured to determine the probability of a diagnosis of an ophthalmological condition, or of a plurality of ophthalmological conditions.

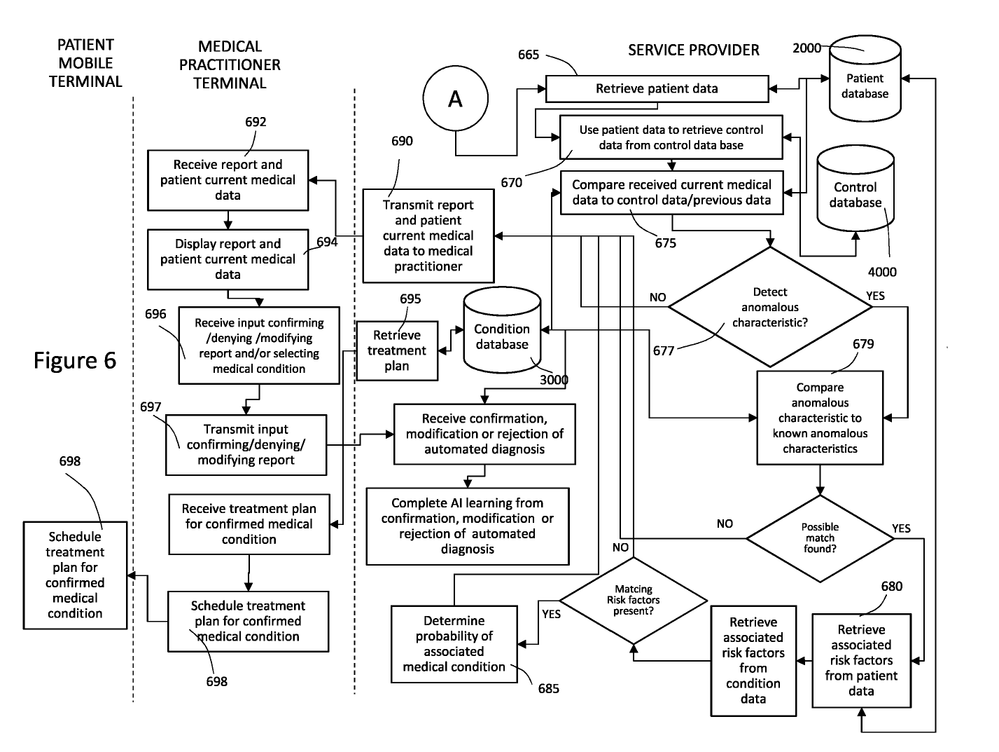

An exemplary decision-making process that is carried out in the diagnosing of an ophthalmological condition is shown in more detail in FIG. 6.

In this regard, the service provider system facilitates access to a condition database. The condition database can be part of the service provider system, or it can be a third-party system, such as a database held by an insurance provider. Alternately, it is envisaged that the condition data base could be a database that is created by assimilation of anonymous medical records by for example a supercomputer making use of artificial intelligence techniques such as Bayesian networks, neural networks, machine learning, evolutionary computation, fuzzy systems chaos theory or the like.

In order to determine whether an ophthalmological condition is present, the service provider system 200 will initially determine whether there is an anomalous characteristic in the current medical data. An example of an anomalous characteristic could be the presence of a lesion, cavity or recess on the patient's retina. In order to detect the anomalous characteristic in a given set of patient's current medical data, patient properties are retrieved 665 from the patient details (such as age, weight, race, gender, etc.), and a control database is interrogated 670 using the patient properties, to retrieve a set of control data.

The set of control data that is retrieved is data that is similar in nature to the current medical data that has been received (e.g., if the patient's current medical data is a 3D scan of the patient's eye, then a 3D scan of a healthy patient's eye will be retrieved). The control data will also be matched for healthy patients having the same or a similar set of patient properties such as age, weight, race, gender, eye color or the like.

It is envisaged that the set of control data will be held in a control database 4000, and will be ordered according to the data being compared, as well as the properties being controlled for or taken into account.

Once the set of control data has been retrieved 670, the patient's current medical data is compared 675 to the control data to detect 677 if there are any anomalous characteristics in the patient's current medical data. In this way the control data is used as a filter to filter out anomalies in the current medical data.

Once an anomalous characteristic is detected 677 in the patient's current medical data, then this anomalous characteristic is used to interrogate 679 the condition database to compare 679 the detected anomalous characteristic to the record of known anomalous. Examples of an anomalous characteristic could include a visual pattern detected in an image, a shaped recess in the 3D structure of a patient's retina, a low pressure reading on an intraocular pressure test, or any such anomaly in the data.

It is envisaged that the condition database will include a list of anomalous characteristics, with each anomalous characteristic being associated with one or more ophthalmological conditions. Further, each ophthalmological condition is associated with a set of risk factors. The risk factors are factors that increase the likelihood of the diagnosis of that ophthalmological condition for a given anomalous characteristic.

The condition data base preferably further includes best practice treatment plans and treatment schedules for each of the ophthalmological conditions. The condition database further includes associated information such as symptoms, tests and indications for the stored ophthalmological conditions, which can be used by a medical practitioner to confirm a diagnosis of a medical condition.

If a similar anomalous characteristic is found on the condition data base, then the associated medical or ophthalmological condition is retrieved, as well as the risk factors associated with them. The system will retrieve 680 risk factors associated with the patient from the patient data, including their medical history data and their family medical history data. The risk factors will have an associated weighting, which will also be retrieved. The weighting is a factor that is indicative of how much the presence of that risk factor affects the likelihood that the detected anomalous characteristic is indicative of the associated ophthalmological condition.

The patient's risk factors found in their patient data will be tested against the risk factors retrieved from the conditions database to find matches. If the patient risk factors match the retrieved risk factors, then the weightings associated with each of the risk factors is used to determine 685 a probability that the anomalous characteristic detected from the patient's current medical data is indicative of the medical condition retrieved from the condition database.

The retrieved ophthalmological condition(s), preferably together with the determined probabilities for each, are then transmitted 690 to a medical practitioner for presentation, or made available to a medical practitioner over a website, for them to assess. In a preferred embodiment, when the most probable diagnosed ophthalmological conditions are received 692 by and presented 694 to the medical practitioner by display on the medical practitioner's terminal, they are presented together with the facts retrieved from the current medical data and the patient details as support for the diagnosis and determined probability. In this way, a case is made out to the medical practitioner as why a diagnosis was arrived at, and allowing the medical practitioner to verify the diagnosis in a convenient manner.

It is further envisaged that the facts supporting the identification of the condition (i.e., the risk factors detected in the patient data that corresponds to the risk factors associated with the retrieved ophthalmological condition) will be presented in a manner that allows for those facts to be checked, for example by providing the facts with a drop down menu or a hyperlink that allows the medical practitioner to click on it and review the data together with other patient data that may be relevant to the diagnosis or the probability of that diagnosis.

Further, it is envisaged that the most probable diagnosed ophthalmological conditions can be presented 694 to the medical practitioner, together with recommendations for additional tests that and be carried out to confirm and/or rule out the diagnosis.

It is envisaged that once the automated diagnosis is presented to the medical practitioner (for example on a remote terminal at their offices, or to a participating medical practitioner located at the service provider offices), the medical practitioner will review the automated diagnoses that the service provider system has determined, as well as the probabilities for each, and the facts on which the diagnoses are based.

The medical practitioner then has the option of providing input 696 confirming, rejecting or modifying any of the automated diagnoses, and will be provided with means to input why a diagnosis was rejected. The confirmation, rejection or modification will then be transmitted 697 back to the service provider system 200, for use in further training the service provider system to provide better identification of medical conditions in the future. In this way, the system receives feedback that will allow for better identification of medical conditions in the future, either by providing for better identification algorithms that can be supplemented by artificial intelligence, or by providing databases with better information.

Further, information provided by patients at their first appointment, and subsequent follow-up appointment (the information relating to relating to their medical histories, their treatment regimes, the symptoms that they are subject to, as well as the severity of their symptoms) can be used for assessment and modification of treatment regimes. The information provided in this way is similar to information obtained from clinical trials, and can be valuable for the ongoing development of treatment regimes for patients of particular demographic groups, for example.

It is envisaged that artificial intelligence type learning can be provided for retraining the system. A discussion of the various artificial intelligence type learning processes would be appreciated by those of ordinary skill in the art and is therefore omitted for simplicity.

### Scheduling and Management of Condition

Once the one or more diagnoses have been confirmed by the medical practitioner, the service provider system will interrogate the condition database to retrieve 695 a treatment plan or testing plan which will preferably include best practice treatment regimes and/or testing regimes and schedules for treatment or testing. These regimes and schedules are then transmitted and presented to the medical practitioner for confirmation or amendment. In this way, the service provider system 200 determines a management plan for the confirmed diagnosed ophthalmological condition.

It is envisaged that the service provider system 200 will be further provide with scheduling instructions configured for scheduling 698 treatment and/or testing of the patient, as well as follow-up visits. The scheduling instructions could also be configured for scheduling testing and/or treatment and/or follow-up visits with other medical practitioners, such as general practitioners.

A table of the steps that will be typically carried out, as well as a listing of the steps will be carried out by, and the equipment on which the steps will be carried out on, is shown below as an example of a typical treatment process:

| Step Description | Performed by | Equipment Required |
|---|---|---|
| Patient either self presents (acute | Automated if follow up appointment. | Big Picture CRM |
| problem) or for routine follow up is | | |
| emailed various available appointment | | |
| times | | |
| Confirms appointment time | Patient | Any internet enabled device |
| Texted/Emailed reminder | Automated both 2 weeks and 2 days | Big Picture CRM |
| | before appointment | |
| Visits tele ophthalmology site (could be | Patient | Appointment pre-registered on |
| located in GP practices, Optometry | | BPEH Ipad App |
| practices, Emergency departments or | | |
| hospitals without specialist Ophthalmic | | |
| cover onsite) | | |
| Self registers | Patient | BPEH Ipad App |
| Privacy Policy consent | Patient | BPEH Ipad App |
| Informed Consent | Patient | BPEH Ipad App |
| Education & Research consent | Patient | BPEH Ipad App |
| Medicare Number/Payment | Patient | BPEH Ipad App +− Paypal |
| Provision (or confirmation) of existing | Patient | BPEH Ipad App |
| GP, Endocrinologist, nephrologist, | | |
| other healthcare providers | | |
| Presenting complaint questions | Nurse/GP/Technician | BPEH Ipad App |
| (structured) IF ANY | | |
| History of Presenting Complaint | Nurse/GP/Technician | BPEH Ipad App |
| (structured S/Sx depending on PC) IF | | |
| ANY | | |
| Medical History (Structured) (provision | Nurse/GP/Technician | BPEH Ipad App |
| or confirmation) | | |
| Surgical History (Structured) (provision | Nurse/GP/Technician | BPEH Ipad App |
| or confirmation) | | |
| Current Medications (structured) | Nurse/GP/Technician | BPEH Ipad App |
| (provision or confirmation) | | |
| Family Hx (Structured) (provision or | Nurse/GP/Technician | BPEH Ipad App |
| confirmation) | | |
| Social Hx (Structured including | Nurse/GP/Technician | BPEH Ipad App |
| previous trauma) (provision or | | |
| confirmation) | | |
| Recommended tests based on Patient | Automatically Provided by Ipad | BPEH Ipad App |
| History | | |
| Visual Acuity Assessment (inc pinhole) | Nurse/GP/Technician | ETDRS Chart/Pinhole. (Results |
| | | manually entered onto BPEH Ipad |
| | | App). Possible alternative = |
| | | patient administered visual acuity |
| | | test (Eg Moptim VAT-200 device if |
| | | medically validated) Or Pearse's |
| | | binocular OCT VA testing |
| Autorefraction | Nurse/GP/Technician | Autorefractometer (eg Welch Allyn |
| | | SpotVision) |
| Pupils Dilated | Nurse/GP/Technician | Rx |
| Colour Fundus Image(s) (? ultrawide) | Nurse/GP/Technician | Eg Zeiss Handheld Visuscout 100 |
| | | (an option for ultrawide could be |
| | | BPEH's image stitching |
| | | technology). |
| OCT Scan Protocol to be Determined | Nurse/GP/Technician | Eg Moptim Mocean 3000+ |
| IOP | Nurse/GP/Technician | Tonometer |
| Other imaging? | Nurse/GP/Technician | Digital image of external eyes |
| | | taken with Ipad? Angiograms? |
| | | B-scans if media opacity? |
| | | (?technician trained to perform) |
| | | OCT angiograms? |
| Other Tests | Automated API integration with | Blood tests |
| | pathology and other imaging | Other imaging tests |
| | databases | |
| Visual Fields | Nurse/GP/Technician | Eg Humphrey HFA II |
| Upload Files to Cloud | Nurse/GP/Technician | BPEH Ipad App and BPEH |
| | | Dynamic Router |
| Remote Review of Scans to assess | Ophthalmologist | BPEH Cloud Based Scan Review |
| adequacy (QA/QI) and high level | | Platform |
| review for Emergency Pathology | | |
| Notification to Nurse/GP/Technician | Ophthalmologist | BPEH Cloud Based Scan Review |
| of QA/QI (via ipad) | | Platform + Ipad |
| If QI repeat specific scan | Nurse/GP/Technician | Depends which scan is identified |
| | | as QI |
| If QA patient advised can depart facility | Nurse/GP/Technician | |
| Remote review and structured | Ophthalmologist | BPEH Cloud Based Scan Review |
| reporting of scans | | Platform |
| Remote review of structured patient | Ophthalmologist | BPEH Cloud Based Scan Report |
| history including S/Sx, VA, | | and Referral Manager Platform |
| Autorefraction, Reported Scans | | |
| Remote generation (and electronic | Ophthalmologist | BPEH Cloud Based Scan Report |
| signing) of structured patient report and | | and Referral Manager Platform |
| referral letter including follow up plan | | |
| recommendations | | |
| Email link to the patient portal for the | Automated | BPEH Cloud Based Scan Report |
| report to patient. | | and Referral Manager Platform + |
| | | CRM + BPEH Patient Portal |
| Email link to the clinician portal to allow | Automated | BPEH Cloud Based Scan Report |
| patients healthcare providers to view all | | and Referral Manager Platform + |
| reports and the comprehensive set of | | CRM + BPEH Clinician Portal |
| scans. | | (+−automated link with GP & |
| | | Specialist EMR programmes*) |

The above table describe exemplary steps carried out in diagnosing an existing condition, however it will be appreciated by a person skilled in the art that similar steps could be carried out as part of a screening function, where a patient had not yet become aware of an existing condition. Further, it will also be appreciated by those skilled in the art that less detailed steps can also be possible by healthcare professionals that are not licensed to carry out some of the steps shown above and.

### EXAMPLES

Examples of the testing and diagnosis of a particular ophthalmological condition are provided below. The specific ophthalmological condition is a Purtscher Retinopathy (PR) and Purtscher Like Retinopathy (PLR).

The diagnostic criteria for a PR is at least three of five criteria, namely:
Purtscher flecks
Retinal hemorrhages, in low to moderate number
Cotton wool spots (confined to the posterior Paul)
Probable explanatory etiology
Complementary investigation compatible with diagnosis

The table below shows an example of patient history that are indications for PR or PLR, as well as image characteristics that indicate a diagnosis of PR or PLR, and further provides additional tests that could be recommended to positively establish the diagnosis of PR or PLR.

| Patient History | Image Characteristics | Additional Tests to establish Dx |
|---|---|---|
| HPC: PR-severe head trauma | Multiple Cotton Wool Spots | Amylase/Lipase (PLR is often associated |
| or crush injury to the chest or | (CWS) (93% cases) and, or | with acute pancreatitis) |
| lower extremities (eg road | superficial hemorrhages in a | Activated complement C5a is associated with |
| traffic accident or CPR). PLR - | peripapillary configuration | the development of PLR in numerous |
| most common cause of PLR | (65% cases), Purtscher | conditions. |
| is acute pancreatitis | flecken areas of inner retinal | EUC |
| (abdominal pain). Other | whitening (63%) Optic disc | ANA lupus (>95% positive) and scleroderma |
| causes of PLR pancreatic | edema. | (about 40% positive). Dermatomyositis rarely |
| adenocarcinoma/renal failure/ | Typically bilateral (60% PR | shows a positive ANA. |
| preeclampsia & childbirth | cases) but can be unilateral | Anti-ds DNA antibody is frequently positive in |
| PMHx: Unexplained vision | and asymmetric | lupus (>75% positive) and scleroderma |
| loss in patients with SLE, | Virtually all cases of PLR | (about 15-50% positive). |
| dermatomyositis, scleroderma | precipitated by acute | Rheumatoid factor is positive in |
| should raise the possibility of | pancreatitis are bilateral | approximately 40% of patients with |
| PLR. | Location: Most of the cases | dermatomyositis. |
| Sx Hx: Orthopaedic Sx (fat | (⅔rd) involve zone A of the | Elevated serum transaminase/creatine |
| embolisation) | retina alone. Zone C is | phosphokinase/serum aldolase/myoglobin |
| Social Hx: Valsalva | typically not involved. | Elevated urine myoglobin (Evidence of |
| maneuver/weight-lifting. | OCT (Acute Phase) may | muscle breakdown with dermatomyositis) |
| Significant alcohol | show a hyperreflectivity in | Visual Fields: central, paracentral or arcuate |
| consumption (a common | the inner retinal layers | scotoma. Peripheral visual field is usually |
| cause of pancreatitis) | corresponding to cotton-wool | preserved. |
| Signs & Symptoms: | spots and a variable degree | FA blocked choroidal fluorescence (either due |
| diminished visual acuity | of macular edema. | to retinal whitening or blood), occluded retinal |
| (typically with visual field loss). | OCT (Late/chronic) variable | arterioles, areas of capillary non-perfusion, |
| In PR, visual disturbance | degree of outer retinal | late leakage from the retinal vessels in areas |
| may appear synchronous with | atrophy and photoreceptor | of ischemia and optic disc edema. Early acute |
| trauma or be delayed up to | loss | changes (within 2 hours) show slight early |
| 24-48 h. Typically in the range | OCT: Paracentral acute | masking of choroidal fluorescence in the |
| of 20/200 to counting fingers. | middle maculopathy | affected area, with subsequent arteriolar |
| Vision often improves over | characterised by hyper- | leakage. Leakage from the optic nerve has |
| several months to a range of | reflectivity at the inner | also been reported |
| 20/30 to 20/200, depending on | nuclear layer which signifies | Multi-focal ERG - a depression in both A- |
| the severity of the retinal | involvement of deep and | waves and B-waves in the affected retina. |
| findings. | intermediate retinal plexuses | |

The following table sets out pathology image characteristics for identification of Cotton Wool Spots (CWS) in an asymptomatic patient:

| Image Type | Characteristic (* - Location) |
|---|---|
| Colour Fundus Image | Whitening in the superficial retinal nerve fibre layer |
| OCT Tomogram | At presentation, initially increased retinal thickness with focal thickening of the retinal nerve fiber |
| | layer (seen on tomograms) |
| | Hyper-reflective pattern that persists even after they become ophthalmoscopically invisible |
| OCT Retinal | At presentation showing thickening of an area corresponding to the CWS. |
| Thickness Maps | After “resolution” showing focal thinning on the retinal thickness map (compared with the adjacent |
| | healthy retina). |

A differential diagnosis (i.e., alternative diagnoses) could be for Myelinated Nerve Fibre Layer, or retinal whitening secondary to neuro retinitis. The etiology or cause of Cotton Wool Spots are thought to be as a result of an acute obstruction of a pre-capillary retinal arteriole causing blockage of axoplasmic flow and buildup of axoplasmic debris in the nerve fibre layer (NFL).

The presence of CWS can be indications for the following ophthalmological conditions:
The presence of CWS are useful signs for grading hypertensive retinopathy and diabetic retinopathy.
CWS signals a declining CD4 count in HIV disease.
CWS are commonly seen in the course of central and branch retinal vein occlusions.
CWS can be a presenting sign of multiple myeloma and post radiation retinopathy.
CWS are associated with a wide spectrum of diseases including: cardiac valvular disease, Purtscher retinopathy, corrected artery obstruction, dermatomyositis, systemic lupus erythematosus, polyarteritis nodosa, leukaemia, lymphoma, metastatic carcinoma, and giant cell arteritis

The various ophthalmological and medical conditions mentioned above would be stored in the conditions database in association with CWS.

Using the above information, a patient who is male, 45 years old and having diabetes, would register with the service provider system, and attend a remote input terminal 100, where tests would be carried out and scans taken of their eyes. During this process, patients would also input patient details, including presenting complaint, history of presenting complaint, historical medical details and family historical medical details.

Scans taken of their eyes could for example include colour fundus image scans, OCT tomogram scans and OCT retinal thickness mapping. Examples of testing that may be carried out include visual acuity measurements, auto refraction measurements and/or intraocular pressure tests. Control scans of healthy comparable patients (e.g., male, 45-year-old Caucasian patients) will be retrieved from the control database. The patient scans would be compared to the retrieved control scans. From this comparison, an anomalous characteristic would be detected. The anomalous characteristic is compared to all of the anomalous characteristics on the condition database by comparison of the visual images, to retrieve a known anomalous characteristic that has a best fit to the detected anomalous characteristic. In this case, the best fit to the detected anomalous characteristic would be Cotton Wool Spots.

It is further envisaged that the patient scans and/or tests will be compared to previous historical tests and scans for that patient. In this way, changes in the results for a particular patient can be picked up as anomalous characteristics. It is envisaged that not just scans could be tested against previous scans, but test results for visual acuity and auto refraction, or any other test results.

Cotton Wool Spots (CWS) are associated with several ophthalmological conditions on the condition database—for example PR, PLR, leukaemia, lymphoma, diabetes, et cetera. The ophthalmological condition is in turn also associated with risk factors that increase the probability of that anomalous characteristic being as a result of influence of a particular ophthalmological condition. All of the associated risk factors are retrieved from the condition database. Further, the patient details are checked to if matching risk factors are present.

In this exemplary case, the patient's medical history of diabetes has a strong associated weighting factor that the presence of CWS is due to diabetic retinopathy. Additional risk factors may also strengthen this probability where the risk factors are cumulative. Each of these risk factors have weighting associated with it, and the weighting is used to calculate the increased probability of the diagnosis of that ophthalmological condition. For this example, a patient medical history of having diabetes would match to the associated risk factors in the condition database, and the weighting would be used to increase the probability of diabetic retinopathy as being the diagnosed condition. This diagnosis may in turn be strengthened by the presence of additional factors such as smoking, age, et cetera. Similarly, if a patient medical history showed the presence of cancer, then the probability of the CWS being an indicator for post radiation retinopathy increases.

The presence of two or more anomalous characteristics also be used to increase or decrease weighting factors. For example, if cotton wool spots were present alone, and the patient showed a medical history of diabetes, then this may weigh the diagnosis towards diabetic retinopathy. However, if additional anomalous characteristic such as Purtscher flecks and/or retinal hemorrhages in low to moderate numbers were present, then the presence of these additional anomalous characteristics will increase the weighting given to the diagnosis of PR or PLR.

The establishment of a diagnosis is an important step for both the clinician and the patient. However, the establishment of a specific diagnosis does not lead immediately to a known treatment/management plan and prognosis. Similar to establishing a diagnosis as described above, the treatment plan and resultant prognoses are subject to contextual variation based on such factors as patient demographics, history, investigative tests results, etc. The treatment plan is preferably personalised or customized for each patient. Structured clinical history can be used to assist the clinician in determining the most appropriate treatment plan for the individual patient and the resultant prognosis. Examples of contextual clinical history that are relevant in determining a personalised treatment plan and prognosis are outlined in the table below:

| | | Impact on treatment/ |
|---|---|---|
| | | management plan/ |
| Diagnosis | Contextual Questions | Prognosis |
| Patient diagnosed with | Do you wear contact lenses? | The platform will recommend |
| a bacterial corneal eye | No | (1) first line empirical |
| infection without | Are you a diabetic? No | treatment for non-contact |
| ulceration | Allergies to medications? Yes | lens wearing corneal |
| | Which Medication? Penicillin | infection that is non-penicillin |
| | Allergic response? | based eg a Fluoroquinolone |
| | Anaphylaxis | such as ciprofloxacin. (2) |
| | | follow up with a general |
| | | practitioner or Emergency |
| | | department only if there is |
| | | any deterioration in vision, |
| | | severe eye pain and/or |
| | | worsening of infective |
| | | symptoms. |
| | | Platform will also provide a |
| | | prognosis based on the |
| | | diagnosis, structured clinical |
| | | history, patient |
| | | demographics, and |
| | | recommended treatment |
| | | plan. In this case, the |
| | | infection is likely to resolve |
| | | without further intervention. |
| | | More specific quantification |
| | | can even be quantified by |
| | | the platform with the |
| | | introduction of AI. |
| Patient diagnosed with | HPC: Do you wear contact | Patients that wear contact |
| a bacterial corneal eye | lenses: Yes | lenses are at a much higher |
| infection with ulceration | HPC: Have you had any | risk of developing severe |
| <2 mm | associated reduction in your | keratitis (an infection of the |
| | vision? Yes | cornea). The co-morbidity of |
| | PMHx: Are you a diabetic? | diabetes further increases |
| | Yes | this risk of severe infection. |
| | Allergies: Allergies to | The platform will recommend |
| | medications? No | (1) that the patient |
| | Social Hx: Is the patient | immediately cease wearing |
| | independent with their | the contact lenses (2) |
| | activities of daily living? Yes | Suggest immediate empirical |
| | | treatment with a topical |
| | | aminoglycoside antibiotic |
| | | such as tobramycin 0.3% (3) |
| | | Patient requires follow up |
| | | the next day with an |
| | | Ophthalmologist. Note, if the |
| | | contextual question relating |
| | | to activities of daily living, |
| | | was “No”, then this patient |
| | | would be immediately |
| | | admitted to hospital to |
| | | ensure compliance with the |
| | | treatment plan. |
| | | The Platform will also |
| | | provide a prognosis based |
| | | on the diagnosis, structured |
| | | clinical history, patient |
| | | demographics, and |
| | | recommended treatment |
| | | plan. In this case, in the |
| | | absence of treatment, there |
| | | is potential for rapid |
| | | devastating loss of vision. |
| | | With the introduction of AI, |
| | | the platform would also be |
| | | able to provide the |
| | | probability of full resolution |
| | | in a particular time frame for |
| | | the patient. |

A further table illustrating different factors and features for diagnosis and treatment is shown in Appendix A.

### Virtual Reality Simulation System

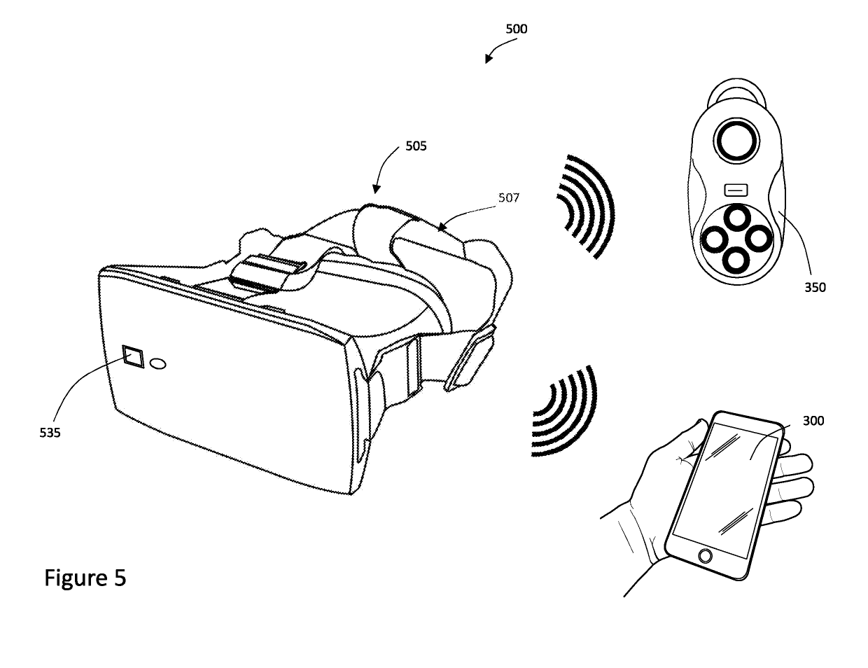

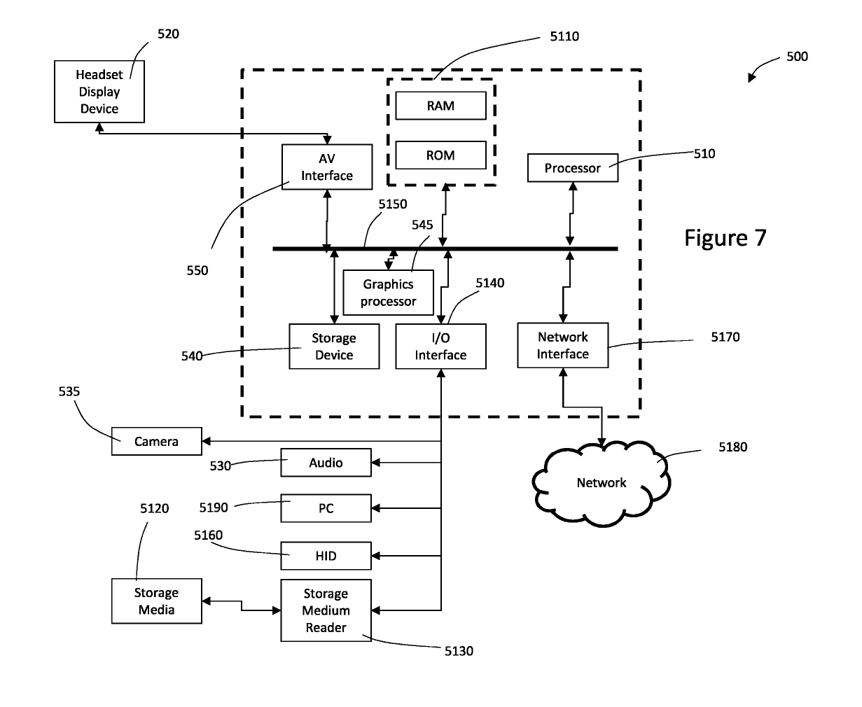

In a further aspect, and as shown in FIGS. 5 and 7, there is provided a simulation system 500 for simulating an ophthalmological condition. In FIG. 10 similar features to those shown in FIG. 1 are provided, with a numeral “5” prefacing the numerals compared to those of FIG. 1. However, it is envisaged that the simulation system 500 can further include a graphics processor 545 for use in processing the image processing filters or shaders as described below.

The system includes a camera 535 configured to transmit a digital visual image or visual image stream (hereinafter the “visual image”), a wearable virtual reality headset 505 configured to display the visual image to a user on which the headset is mounted, a processor 510 for processing digital information and instructions, and digital storage media 540 for storing instructions. The digital storage media includes instructions for instructing the processor to process the visual image received from the camera as described below.

The virtual reality headset 505 includes a headset display 520, and a mounting arrangement 507, preferably including webbing, for mounting the headset on to a user's head.

The system 500 will include a database of ophthalmological conditions, such as pathologies and/or eye conditions. Each of the ophthalmological conditions is associated with at least one or more image processing filters or shaders.

The system is configured for receiving a condition selection input, preferably from a user, selecting an ophthalmological condition, for example by selection from a drop-down menu on a touch enabled screen, or any other suitable input device such a as a keyboard or mouse. On receipt of the input identifying the ophthalmological condition, the system will retrieve the at least one or more image processing filter, or shader, associated with that ophthalmological condition. The image processing filters will be retrieved from the digital storage media, or over a network such as the Internet.

The visual image received from the camera will be processed to be displayed for viewing on the virtual reality headset, so that the user on which the headset is mounted will see the visual image received from the camera. On selection of the ophthalmological condition, preferably by the user, one or more image processing filters will be retrieved from the database that are associated with that ophthalmological condition. The image processing filters will then be used to process the visual image received from the camera, and the processed visual image will then be displayed on the virtual reality headset. The processed visual image will be indicative of how the selected ophthalmological condition will affect the vision of a person having that ophthalmological condition.

The system will also similarly preferably be configured for receiving a severity selection input, preferably from a user, selecting a level of severity of the ophthalmological condition. On receipt of the severity selection input, the system retrieves associated modifiers associated with the ophthalmological condition on the condition database, which will instruct modification of the visual image in accordance with the selected severity selection input. It is envisaged that, for example, the maximum level of severity would correspond to the processed visual image displaying an extreme effect of the selected ophthalmological condition on a user's vision, while the minimum level of severity would display the effect of a mild form of the ophthalmological condition.

In one embodiment, a severity selection input is not required, and a shadow or image processing filter can be selected and applied without requiring a severity selection input.

In a preferred embodiment, the level of severity will be passed as a floating point value that is passed to the ophthalmological condition shader, which uses the floating point value to determine (via mathematical functions) the parameters to use for the image processing filters, and/or which image processing filters to use.

In this way, a user can the educated on the effect and potential effect of an ophthalmological condition on their vision. Further, it is envisaged that, by cycling through the various effects of ophthalmological conditions, a user can select a processed visual image as one that most closely resembles the effect of an ophthalmological logical condition on their own vision. This can aid a user or patient in explaining to a medical practitioner what they are seeing in their own vision, and the severity thereof.

It is further envisaged that the will be configured for outputting an audio signal that can announce the ophthalmological condition being displayed to the user on the headset display, and can further announce the severity level. Alternatively, the ophthalmological condition and/or the severity thereof can be displayed on the display itself.

Examples of ophthalmological conditions that can be simulated by the image processing filters include: Cataract; Glaucoma; Refractive conditions (e.g., Myopia, Hyperopia, Astigmatism, and Presbyopia); Other macula conditions (e.g., Age-related Macula Degeneration, Macula hole, Macula Oedema, and Vitreomacula traction); Retinal conditions (e.g., Diabetic Retinopathy, Retinal detachment, Artery and Vein Occlusions, and Vitreous Haemorrhage, Central Serous Retinopathy, Epiretinal Membrane, Retinitis Pigmentosa, Colour vision defects, and Retinal Hole); Flashes; Floaters; and Neuro-ophthalmology (e.g., Visual consequences of neurological disease, Visual field defects (e.g., Hemianopia, Quadrantinopia), Visual migraine/aura, Amaurosis Fugax, Transient Ischaemic Attack, Visual disturbances, and Double vision).

In a preferred embodiment, the algorithm for simulating each condition is implemented as a graphics “shader” written in the GLS allocating language used by OpenGL, although alternative coding could be used to work with different shading languages.

It is envisaged that different ophthalmological conditions could use a combination of shaders, so that the combined effect of the shaders or filters will mimic the ophthalmological condition.

Examples of various shaders that are envisaged include:
A shader that can capture input from the camera and process it to transform it from the wide UV colour space into the RGB colour space.
A blurring shader that can decrease or eliminates the high-frequency content of the full image or parts of the image.
A bloom shader that can be used to enhance and spread out bright light sources (i.e., to simulate glare).
A shader that applies a custom point spread function.
A shader that applies a custom point spread function, for example Zernike polynomials.
A shader that makes a part of the image darker or lighter or more like a certain color (e.g., grey).
A shader that decreases the saturation of part of the image,
A shader that changes the hue of part of the image.
An inpainting shader that replaces parts of an image by color values computed from other parts of the image.
   As example of the effect that this inpainting shader is simulating, when somebody has a blind spot, or even a stroke affecting their vision, this typically does not actually appear as a black area to the person. Instead their brain attempts to create a best guess at filling in what the eyes do not see—conceptually the inpainting shader works in two steps: first a part of the image is made black, i.e., the image information in that part is removed, and then the information in the removed part is filled in with colour or other information from the parts of the image that were not removed;
A shader that distorts the image by applying translations, rotations, scalings, and/or general free-form warping functions to the image.
A shader that adds random noise in different shapes and sizes to the image, for example by using a Perlin simplex noise implementation.
A ghosting shader that copies part of or all of a previous image, possibly alters it using any of the above shaders, and pastes it onto the current image.

It is further envisaged that any of the image processing filters can be configured to change display over time. For example, the floaters shader can simulate dark or bright spots moving through the display over time in various directions of movement, or in patterns of movement.

The system can then take the processed visual image and transform it into two separate images, one for what the left eye sees and one for what the right eye sees. Each of these images are then presented on the display of the headset.

It will be appreciated from the above explanation that any visual processing filter that may be applied can be a combination of any other visual processing filters.

In a preferred embodiment, it is envisaged that a remote control device 350 can be provided that is configured to control the ophthalmological conditions that are selected, and the severity thereof. Where, for example the virtual reality headset is a dedicated headset with its own display, it is envisaged that the remote control device can be a mobile electronic device such as a smart phone 300, onto which a control application has been downloaded. The smartphone can be connected to the virtual reality headset by a wired or wireless connection, to control operation of the headset display. It is further envisaged that the headset display can be replicated on the smartphone 300 display, so that the person controlling what is being displayed on the headset (such as a medical practitioner, GP or the like) will be aware of what is being displayed on the headset.

In an alternative embodiment, the smartphone itself is inserted into a head mount to be used as a headset. In this embodiment, it is envisaged that the microphone can be controlled by a dedicated remote device 350 that can be connected to the smartphone by a cable or wirelessly. It is further envisaged that another smartphone can be connected to the smartphone to control the display on the headset.

In this way, it is envisaged that levels of compliance with treatment regimes by patients can be increased. Also, by being able to display the potential effects of non-compliance with examinations and scans as suggested by clinicians, patients will be more likely to follow up with later appointments.

Additionally, it is envisaged that more accurate identification of ophthalmological conditions, pathologies and eye conditions will be possible, as well as more accurate assessment of their severity, without reliance on subjective descriptions of symptoms from patients.

It will be appreciated by those skilled in the art that in one embodiment, the headset may merely act as a receiver for receiving visual images from a camera, and transmitting them to a remote terminal for processing, and then receiving signals from a remote terminal, and displaying them on the headset display.

In another embodiment, the headset can include a processor, as well as the database of ophthalmological conditions and/or associated medical image filters, and will be able to receive visual images from the camera, process the visual images and display them on the headset in a suitable format, for example as a pair of images.

In yet another embodiment, the headset can receive the visual images from a camera, while the ophthalmological condition is selectable by a remote device 350, and the associated image processing filters are retrieved from a remote database and transmitted to the headset, where a processor processes the received visual images using the received image processing filters to present them in a suitable format as processed images.

A systematized way to take a patient history, perform an examination, form a differential diagnosis and then order tests to confirm the diagnosis and subsequently establish an appropriate treatment plan is described above. Although embodiments and examples have been described in relation to the ophthalmic field, it will be appreciated that many of the features described above are applicable to other areas of medicine. By way of example only:

### (A) Dermatological Problem:

Patient presents to a General Practitioner with a skin lesion they are concerned about The GP (or nurse practitioner):
i. Takes a highly focused and structured patient history (Presenting complaint, past medical history, medications, allergies, social history etc.) using the previously described system.
ii. Takes a digital image of the skin lesion e.g., with a digital camera and/or digital dermatoscope (this could be supplemented by an OCT of the skin lesion).
iii. Depending upon the responses to the contextual questions, the previously described system may also suggest further additional tests (e.g., blood tests for tumour markers, x-ray or CAT scan looking for metastases, biopsy or skin scrapings for pathology) that could be recommended to positively establish the diagnosis.
iv. Uploads the information to the previously described system for analysis by its algorithms that subsequently provides a probabilistic diagnosis, together with a personalised treatment/management plan and prognosis that the GP can discuss/implement with the patient.

### (B) Orthopaedic Problem:

Patient presents to the emergency department with a painful right hip. The clinician (or nurse practitioner):
i. Takes a highly focused and structured patient history (Presenting complaint, past medical history, medications, allergies, social history etc.) using the previously described system.
ii. Depending upon the responses to the contextual questions, the previously described system may also suggest tests (e.g., core temperature, blood tests looking for signs of infection, x-ray or CAT scan looking fractures or osteoarthritis, joint aspirate looking for infection) that could be recommended to positively establish the diagnosis.
iii. Uploads the information to the previously described system for analysis by its algorithms that subsequently provides a probabilistic diagnosis, together with a personalised treatment/management plan and prognosis that the clinician can discuss/implement with the patient.

### (C) Cardiology Problem:

Patient presents to the emergency department with chest pain. The clinician:
a) Takes a highly focused and structured patient history (Presenting complaint, past medical history, medications, allergies, social history etc.) using the previously described system.
b) Depending upon the responses to the contextual questions, the previously described system may also suggest tests e.g., ECG looking for signs of ischaemic heart disease (heart attack), chest x-ray looking for a collapsed lung, troponin blood tests looking evidence of damage to the heart muscle, d-dimer blood test to rule out the likelihood of a clot in the lungs, CAT scan with contrast looking for a dissection of the aorta etc that could be recommended to positively establish the diagnosis.
c) Uploads the information to the previously described system for analysis by its algorithms that subsequently provides a probabilistic diagnosis, together with a personalised treatment/management plan and prognosis that the clinician can discuss/implement with the patient.
   It will be appreciated that the steps described may be performed in a different order, varied, or omitted entirely without departing from the scope of the present disclosure.
   The features described with respect to one embodiment may be applied to other embodiments, or combined with or interchanged with the features of other embodiments, as appropriate, without departing from the scope of the present disclosure.
   Other embodiments of the invention will be apparent to those skilled in the art from consideration of the specification and practice of the invention disclosed herein. It is intended that the specification and examples be considered as exemplary only, with a true scope and spirit of the invention being indicated by the following claims.
   In addition to uses in different specialties there are also extensions into primary health and the home. Capturing family, social and medical history is important but mostly not essential to diagnosis and management plan, and it is time consuming and so detailed history is skipped during consultations. However the patient is motivated to provide all possible information to ensure that the diagnosis is made with all available information. The software allows the patient to log in and update their family, social and medical history from any location as long as they can verify their identity. The questions are answered within a web form that is then available to the technician as part of the history review and to all relevant scan review and diagnoses screens.

### Interpretation

### And/or:

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements).
   In accordance with:
As described herein, “in accordance with” may also mean “as a function of” and is not necessarily limited to the integers specified in relation thereto.

### Specific Details

In the description provided herein, numerous specific details are set forth. However, it is understood that embodiments of the invention may be practiced without these specific details. In other instances, well-known methods, structures and techniques have not been shown in detail in order not to obscure an understanding of this description.

### Chronological Order

For the purpose of this specification, where method steps are described in sequence, the sequence does not necessarily mean that the steps are to be carried out in chronological order in that sequence, unless there is no other logical manner of interpreting the sequence.
   Markush groups
In addition, where features or aspects of the invention are described in terms of Markush groups, those skilled in the art will recognise that the invention is also thereby described in terms of any individual member or subgroup of members of the Markush group.

APPENDIX A

| | | | | | Medicine | |
|---|---|---|---|---|---|---|
| | | | | Medicine | primary | |
| Category | Subcategory | Detail | Ophthalmology | speciality | care | Home |
| Diagnosis | History | Presenting complaint | See app for | Contextual - | General - broad qns | |
| | | | details | based on PC | | |
| Diagnosis | History | History of presenting | See app for | Contextual - | General - broad qns | |
| | | complaint | details | based on PC | | |
| Diagnosis | History | Past presenting | See app for | Contextual - | General - broad qns | |
| | | complaint history | details | based on PC | | |
| Diagnosis | History | Past medical history | See app for | Contextual - | General - broad qns | |
| | | | details | based on PC | | |
| Diagnosis | History | Medications & allergies | See app for | Contextual - | General - broad qns | |
| | | | details | based on PC | | |
| Diagnosis | History | Family history | See app for | Contextual - | General - broad qns | Yes |
| | | | details | based on PC | | |
| Diagnosis | History | Social history | See app for | Contextual - | General - broad qns | Yes |
| | | | details | based on PC | | |
| Diagnosis | Images | Transmission | Query | | | |
| Diagnosis | Images | Compression | Query | | | |
| Diagnosis | Images | Dynamic display/loading | Query | | | |
| Diagnosis | Images | All in one scan review | [168] | | | |
| | | (scan, signs, symptoms, | | | | |
| | | scans) to create structured | | | | |
| | | reporting | | | | |
| Diagnosis | Images | Dataset of | See app for | Extension in | Potential in | Cardiology |
| | | characteristics & | details | application | application | Derm |
| | | abnormalities | | | | Ortho |
| Diagnosis | Tests | Measurements | See app for | Potential in | Potential in | Cardiology |
| | | (Symptoms) | details | application | application | Derm |
| | | | | | | Ortho |
| Diagnosis | Tests | Additional tests | See app for | Potential in | Potential in | Cardiology |
| | | | details | application | application | Derm |
| | | | | | | Ortho |
| Diagnosis | Output | Diagnosis algorithm to | Query | | | |
| | | create probabilities | | | | |
| Diagnosis | Output | Patient diagnosis | Query | | | |
| | | probability | | | | |
| Prognosis | Input | Dataset of characteristics | Query | | | |
| | | & prognoses | | | | |
| Prognosis | Comparative | Al/pattern recognition/ | Query | | | |
| | factors | analysis on data set | | | | |
| Prognosis | Relative | Prognosis algorithm | Query | | | |
| | analysis | | | | | |
| Prognosis | Relative | Communication & | Query | | | |
| | analysis | reporting | | | | |
| Treatment | Input | Structured input of | Query | | | |
| | | diagnosis & prognosis | | | | |
| Treatment | History | Social history | See app for | Contextual - | General - broad qns | Yes |
| | | | details | based on PC | | |
| Treatment | History | Lifestyle requirements | Query | | | Yes |
| Treatment | History | Medications & allergies | See app for | Contextual - | General - broad qns | |
| | | | details | based on PC | | |
| Treatment | Output | Treatment algorithm | Query | | | |
| Treatment | Output | Act - operate, medicate | Query | | | |
| Treatment | Output | Follow up - when, if, how | Query | | | |
| Treatment | Output | Communication & | Query | | | |
| | | automatically generated | | | | |
| | | reporting | | | | |
| Treatment | Output | Communication (letter, | Query | | | |
| | | appointment) integration | | | | |

1. A system for identifying an abnormal medical condition in a patient carried out on an electronic device, the system comprising:
a processor;
a network interface coupled to the processor;
digital storage media operatively associated to the processor, the digital storage media comprising:
an anomalous characteristic detection module configured to receive current medical data relating to the patient, and detect an anomalous characteristic in the current medical data indicative of an anomaly that could be indicative of a medical condition; and
a medical condition determination module configured to compare the detected anomalous characteristic to a database of anomalous characteristics to retrieve an associated medical condition as a determined medical condition.

2. The system as claimed in claim 1, wherein the anomalous characteristic detection module is configured for receiving data from a digital ophthalmological data collection device configured for capturing current medical data relating to a patient's eye.

3. The system as claimed in claim 1, wherein the anomalous characteristic detection module is configured to filter the received current medical data to detect the anomalous characteristic.

4. The system as claimed in claim 1, wherein the anomalous characteristic detection module is configured to filter the patient's received current medical data against medical data of healthy patients.

5. The system as claimed in claim 1, wherein the anomalous characteristic detection module is configured to receive patient data relating to the circumstances of the patient.

6. The system as claimed in claim 5, wherein the received patient data comprises one or more selected from patient historical data; patient historical medical data; and patient family historical medical data.

7. The system as claimed in claim 1, wherein the anomalous characteristic detection module is configured to interrogate a control database that stores personal data of healthy people, and associated baseline medical data of healthy people.

8. The system as claimed in claim 7, wherein the system comprises a control database that stores personal data of healthy people, and associated baseline medical data of healthy people.

9. The system as claimed in claim 7, wherein the anomalous characteristic detection module is configured to interrogate the control database to compare at least one or more of the received patient details to the personal data of healthy people in order to compare like for like medical details, and then retrieving associated medical data of healthy people as a baseline filter to detect an anomalous characteristic in the patient data.

10. The system as claimed in claim 1, wherein the medical condition determination module is configured to access a condition database comprising:
a plurality of medical conditions; and
anomalous characteristics associated with the medical condition.

11. The system as claimed in claim 10, wherein the condition database further comprises risk factors associated with that medical condition, the presence of which increases the likelihood of the anomalous characteristic being indicative of that medical condition.

12. The system as claimed in claim 10, wherein the system comprises a condition database.

13. The system as claimed in claim 10, wherein the medical condition determination module is configured to:
compare at least one or more detected anomalous characteristic of the patient to anomalous characteristics listed in the condition database to detect a match; and
retrieve at least one or more medical condition associated with a matching anomalous characteristic.

14. The system as claimed in claim 11, wherein the medical condition determination module is configured to interrogate the condition database to:
compare at least one or more detected anomalous characteristics to the listed anomalous characteristics;
compare at least part of the information in the patient data to the risk factors associated with the listed anomalous characteristics to detect a matching risk factor; and
retrieve at least one or more medical condition associated with a matching anomalous characteristic.

15. The system as claimed in claim 1, wherein the system comprises an assimilation direction module, the assimilation direction module being configured to directing the assimilation of a condition database of medical conditions, with associated anomalous characteristics and associated risk factors.

16. The system as claimed in claim 15, wherein the assimilation direction module is configured to direct the assimilation of a condition database in a networked supercomputer.

17. The system as claimed in claim 1, wherein the system comprises a reporting module, the reporting module being configured for transmitting a diagnosis signal indicative of the results of the determination of the medical condition.

18. The system as claimed in claim 17, wherein the diagnosis signal comprises information including any one or more selected from: the patient details; the detected anomalous characteristic; the determined medical condition; the determined probability of the detected anomalous characteristic being indicative of the medical condition; the patient details matching the risk factors affecting the determined probability; and risk factors associated with the medical condition.

19. The system as claimed in claim 17, wherein the diagnosis signal comprises information identifying a plurality of possible determined medical conditions, the determined probability of the detected anomalous characteristic being indicative of each of the possible determined medical conditions, and the patient details matching the risk factors affecting the determined probability of each of the possible determined medical conditions.

20-70. (canceled)

71. A method for identifying an abnormal ophthalmological condition in a digital eye scan, comprising:
producing, with a digital ophthalmological camera, the digital eye scan;
passing the eye scan through a digital filter to detect at least one abnormal ophthalmological characteristic;
assigning a weight to each abnormal ophthalmological characteristic detected;
dynamically comparing the weighted characteristics detected with a plurality of characteristics indicative of abnormal ophthalmological conditions; and
generating an ophthalmological condition report based on the dynamic comparison of weighted characteristics with indicative characteristics.

72-88. (canceled)
